SEQUENCE LISTING (50 sequences)

SEQ ID NO: 1 - DNA, 120 bp, Homo sapiens
acatctccatggctgtaccaccttgtcgggtagcttatcagactgatgttgactgttgaatctcatggcaacaccagtcgatgggctgtctgacattttggtatctttcatctgaccatc

SEQ ID NO: 2 - DNA, 117 bp, Homo sapiens
ctgaaggcttgctgtaggctgtatgctgttaatgctaatcgtgataggggtttttgcctccaactgactcctacatattagcattaacagtgtatgatgcctgttactagcattcac

SEQ ID NO: 3 - DNA, 112 bp, Homo sapiens
aagattgtgaccagtcagaataatgtcaaagtgcttacagtgcaggtagtgatatgtgcatctactgcagtgaaggcacttgtagcattatggtgacagctgcctcgggaag

SEQ ID NO: 4 - DNA, 118 bp, Homo sapiens
cgtggctacagagtttccttagcagagctgtggagtgtgacaatggtgtttgtgtctaaactatcaaacgccattatcacactaaatagctactgctaggcaatccttccctcgataa

SEQ ID NO: 5 - DNA, 25 bp, Homo sapiens
tccatggctgtaccaccttgtcggg

SEQ ID NO: 6 - DNA, 25 bp, Homo sapiens
ctgaaggcttgctgtaggctgtatg

SEQ ID NO: 7 - DNA, 25 bp, Homo sapiens
aagattgtgaccagtcagaataatg

SEQ ID NO: 8 - DNA, 25 bp, Homo sapiens
ctacagagtttccttagcagagctg

SEQ ID NO: 9 - DNA, 25 bp, Homo sapiens
gacattttggtatctttcatctgac

SEQ ID NO: 10 - DNA, 25 bp, Homo sapiens
tatgatgcctgttactagcattcac

SEQ ID NO: 11 - DNA, 25 bp, Homo sapiens
ttatggtgacagctgcctcgggaag

SEQ ID NO: 12 - DNA, 25 bp, Homo sapiens
gctactgctaggcaatccttccctc

SEQ ID NO: 13 - DNA, 24 bp, Artificial sequence
  Synthetic oligomer
cccgacaaggtggtacagccatgg

SEQ ID NO: 14 - DNA, 20 bp, Artificial sequence
  Synthetic oligomer
tgataagctacccgacaagg

SEQ ID NO: 15 - DNA, 18 bp, Artificial sequence
  Synthetic oligomer
cccgacaaggtggtacag

SEQ ID NO: 16 - DNA, 18 bp, Artificial sequence
  Synthetic oligomer
ggtggtacagccatggag

SEQ ID NO: 17 - DNA, 19 bp, Artificial sequence
  Synthetic oligomer
tcagtctgataagctaccc

SEQ ID NO: 18 - DNA, 22 bp, Artificial sequence
  Synthetic oligomer
gctacccgacaaggtggtacag

SEQ ID NO: 19 - DNA, 19 bp, Artificial sequence
  Synthetic oligomer
cagatgaaagataccaaaa

SEQ ID NO: 20 - DNA, 21 bp, Artificial sequence
  Synthetic oligomer
gatgaaagataccaaaatgtc

SEQ ID NO: 21 - DNA, 22 bp, Artificial sequence
  Synthetic oligomer
gataccaaaatgtcagacagcc

SEQ ID NO: 22 - DNA, 23 bp, Artificial sequence
  Synthetic oligomer
tagtcagacagcccatcgactgg

SEQ ID NO: 23 - DNA, 22 bp, Artificial sequence
  Synthetic oligomer
cgactggtgttgccatgagatt

SEQ ID NO: 24 - DNA, 23 bp, Artificial sequence
  Synthetic oligomer
cagctctgctaaggaaactctgt

SEQ ID NO: 25 - DNA, 20 bp, Artificial sequence
  Synthetic oligomer
tcacactccacagctctgct

SEQ ID NO: 26 - DNA, 19 bp, Artificial sequence
  Synthetic oligomer
ccattgtcacactccacag

SEQ ID NO: 27 - DNA, 20 bp, Artificial sequence
  Synthetic oligomer
ggaaactctgtagccacgaa

SEQ ID NO: 28 - DNA, 20 bp, Artificial sequence
  Synthetic oligomer
tagccacgaaggtgttaact

SEQ ID NO: 29 - DNA, 19 bp, Artificial sequence
  Synthetic oligomer
agggaaggattgcctagca

SEQ ID NO: 30 - DNA, 22 bp, Artificial sequence
  Synthetic oligomer
ttgcctagcagtagctatttag

SEQ ID NO: 31 - DNA, 23 bp, Artificial sequence
  Synthetic oligomer
agtagctatttagtgtgataatg

SEQ ID NO: 32 - DNA, 22 bp, Artificial sequence
  Synthetic oligomer
tgtgataatggcgtttgatagt

SEQ ID NO: 33 - DNA, 20 bp, Artificial sequence
  Synthetic oligomer
gacatttatcgagggaagga

SEQ ID NO: 34 - DNA, 18 bp, Artificial sequence
  Synthetic oligomer
catacagcctacagcaag

SEQ ID NO: 35 - DNA, 18 bp, Artificial sequence
  Synthetic oligomer
cctacagcaagccttcag

SEQ ID NO: 36 - DNA, 18 bp, Artificial sequence
  Synthetic oligomer
ctagtaacaggcatcata

SEQ ID NO: 37 - DNA, 18 bp, Artificial sequence
  Synthetic oligomer
gtgaatgctagtaacagg

SEQ ID NO: 38 - DNA, 18 bp, Artificial sequence
  Synthetic oligomer
cattattctgactggtca

SEQ ID NO: 39 - DNA, 18 bp, Artificial sequence
  Synthetic oligomer
ctgactggtcacaatctt

SEQ ID NO: 40 - DNA, 18 bp, Artificial sequence
  Synthetic oligomer
aggcagctgtcaccataa

SEQ ID NO: 41 - DNA, 18 bp, Artificial sequence
  Synthetic oligomer
aggcagctgtcaccataa

SEQ ID NO: 42 - DNA, 22 bp, Artificial sequence
  Synthetic oligomer
ctggtaagcatgtgccgcactt

SEQ ID NO: 43 - DNA, 20 bp, Artificial sequence
  Synthetic oligomer
ccgcacttggggtatttgac

SEQ ID NO: 44 - DNA, 21 bp, Artificial sequence
  Synthetic oligomer
ccctggcctagagctggtaag

SEQ ID NO: 45 - DNA, 21 bp, Artificial sequence
  Synthetic oligomer
gtcaaatacacggagcgtggc

SEQ ID NO: 46 - DNA, 21 bp, Artificial sequence
  Synthetic oligomer
gagcgtggcactgcaggaggc

SEQ ID NO: 47 - DNA, 22 bp, Artificial sequence
  Synthetic oligomer
gtccaactcagcttgtcaaata

SEQ ID NO: 48 - protein, 14 aa, Artificial sequence
  Synthetic transport peptide
RXRRXRRXRRXRXX

SEQ ID NO: 49 - protein, 17 aa, Artificial sequence
  Synthetic transport peptide
RXRXRXRXRXRXRXRXX

SEQ ID NO: 50 - protein, 17 aa, Artificial sequence
  Synthetic transport peptide
RXRXRXRXRXRXRXRXX

(19) United States
(12) Patent Application Publication
(10) Pub. No.: US 2008/0199961 A1
(43) Pub. Date: Aug. 21, 2008

(54) ANTISENSE COMPOSITION AND METHOD FOR INHIBITION OF miRNA BIOGENESIS

(75) Inventors: John E. J. Rasko, Castle Cove (AU); Hnin Thanda Aung, Indooroopilly (AU); Richard K. Bestwick, Corvallis, OR (US); Patrick L. Iversen, Corvallis, OR (US)

Correspondence Address: PERKINS COIE LLP, P.O. BOX 1208, SEATTLE, WA, 98111-1208, US

(73) Assignees: AVI BioPharma, Inc., Corvallis, OR (US); Centenary Institute, Newtown (AU)

(21) Appl. No.: 11/845,025

(22) Filed: Aug. 24, 2007

Related U.S. Application Data
(60) Provisional application No. 60/840,139, filed on Aug. 25, 2006.

(51) Int. Cl. C12N 15/00; C07H 21/00

(52) U.S. Cl. 435/455; 536/24.5

(57) ABSTRACT

The present disclosure relates to compounds and methods for inhibiting the formation of miRNAs that inhibit translation of one or more identified proteins. The compounds comprise antisense oligonucleotides targeting the pri-miRNA precursor of miRNAs.

### CROSS-REFERENCE TO RELATED APPLICATIONS

This application claims benefit under 35 U.S.C. § 119(e) to application Ser. No. 60/840,139, filed Aug. 25, 2006, the contents of which are incorporated herein by reference in its entirety.

### TECHNICAL FIELD

The present disclosure relates to compounds and methods for regulating gene expression, in particular, for suppression or inhibition of miRNA biogenesis by use of an antisense oligonucleotide targeting the miRNA.

### BACKGROUND

MicroRNAs (miRNAs) are an abundant class of endogenously expressed, relatively small RNAs that do not encode protein but regulate mRNA translation by binding with imperfect complementarity in the 3′-untranslated region of their target mRNAs. Recent studies have shown that miRNAs represent a significant layer of post-transcriptional control and function as important regulators of a broad range of biological processes in plants and animals. miRNAs comprise a considerable portion of the human transcriptome, and initial estimates of the number of vertebrate mRNAs regulated by miRNAs number in the thousands with as many as 30% of all genes having miRNA targets in their mRNAs (Lewis, Burge et al. 2005). The biological processes either predicted or demonstrated to be regulated by miRNAs include cell growth, development, transcriptional regulation, signal transduction, protein modification, transport, cell proliferation morphogenesis, intracellular signaling cascades, phosphorylation, cell cycle, response to external stimulus, and cell organization (Lewis, Burge et al. 2005).

Modulating the expression of endogenous genes through the miRNA pathway can be a useful tool for studying gene function, human therapies, and other applications. Due to the ability of miRNAs to induce RNA degradation or repress translation of mRNA which encode important proteins, there is a need for novel compositions for inhibiting miRNA-induced cleavage or repression of mRNA translation.

### REFERENCES

Agrawal, S., S. H. Mayrand, et al. (1990). “Site-specific excision from RNA by RNase H and mixed-phosphate-backbone oligodeoxynucleotides.” Proc Natl Acad Sci USA 87(4): 1401-5.

Blommers, M. J., U. Pieles, et al. (1994). “An approach to the structure determination of nucleic acid analogues hybridized to RNA. NMR studies of a duplex between 2′-OMe RNA and an oligonucleotide containing a single amide backbone modification.” Nucleic Acids Res 22(20): 4187-94.

Bonham, M. A., S. Brown, et al. (1995). “An assessment of the antisense properties of RNase H-competent and steric-blocking oligomers.” Nucleic Acids Res 23(7): 1197-203.

Boudvillain, M., M. Guerin, et al. (1997). “Transplatin-modified oligo(2′-O-methyl ribonucleotide)s: a new tool for selective modulation of gene expression.” Biochemistry 36(10): 2925-31.

Cross, C. W., J. S. Rice, et al. (1997). “Solution structure of an RNA×DNA hybrid duplex containing a 3′-thioformacetal linker and an RNA A-tract.” Biochemistry 36(14): 4096-107.

Davis, S., B. Lollo, et al. (2006). “Improved targeting of miRNA with antisense oligonucleotides.” Nucleic Acids Res 34(8): 2294-304.

Ding, D., S. M. Grayaznov, et al. (1996). “An oligodeoxyribonucleotide N3′→P5′ phosphoramidate duplex forms an A-type helix in solution.” Nucleic Acids Res 24(2): 354-60.

Egholm, M., O. Buchardt, et al. (1993). “PNA hybridizes to complementary oligonucleotides obeying the Watson-Crick hydrogen-bonding rules.” Nature 365(6446): 566-8.

Esau, C., S. Davis, et al. (2006). “miR-122 regulation of lipid metabolism revealed by in vivo antisense targeting.” Cell Metab 3(2): 87-98.

Felgner, P. L., T. R. Gadek, et al. (1987). “Lipofection: a highly efficient, lipid-mediated DNA-transfection procedure.” Proc Natl Acad Sci USA 84(21): 7413-7.

Gait, M. J., A. S. Jones, et al. (1974). “Synthetic-analogues of polynucleotides XII. Synthesis of thymidine derivatives containing an oxyacetamido- or an oxyformamido-linkage instead of a phosphodiester group.” J Chem Soc [Perkin 1] 0(14): 1684-6.

Gee, J. E., I. Robbins, et al. (1998). “Assessment of high-affinity hybridization, RNase H cleavage, and covalent linkage in translation arrest by antisense oligonucleotides.” Antisense Nucleic Acid Drug Dev 8(2): 103-11.

Lesnikowski, Z. J., M. Jaworska, et al. (1990). “Octa(thymidine methanephosphonates) of partially defined stereochemistry: synthesis and effect of chirality at phosphorus on binding to pentadecadeoxyriboadenylic acid.” Nucleic Acids Res 18(8): 2109-15.

Lewis, B. P., C. B. Burge, et al. (2005). “Conserved seed pairing, often flanked by adenosines, indicates that thousands of human genes are microRNA targets.” Cell 120(1): 15-20.

Mertes, M. P. and E. A. Coats (1969). “Synthesis of carbonate analogs of dinucleosides. 3′-Thymidinyl 5′-thymidinyl carbonate, 3′-thymidinyl 5′-(5-fluoro-2′-deoxyuridinyl) carbonate, and 3′-(5-fluoro-2′-deoxyuridinyl) 5′-thymidinyl carbonate.” J Med Chem 12(1): 154-7.

Moulton, H. M., M. H. Nelson, et al. (2004). “Cellular uptake of antisense morpholino oligomers conjugated to arginine-rich peptides.” Bioconjug Chem 15(2): 290-9.

Nelson, M. H., D. A. Stein, et al. (2005). “Arginine-rich peptide conjugation to morpholino oligomers: effects on antisense activity and specificity.” Bioconjug Chem 16(4): 959-66.

Stein, D., E. Foster, et al. (1997). “A specificity comparison of four antisense types: morpholino, 2′-O-methyl RNA, DNA, and phosphorothioate DNA.” Antisense Nucleic Acid Drug Dev 7(3): 151-7.

Summerton, J. and D. Weller (1997). “Morpholino antisense oligomers: design, preparation, and properties.” Antisense Nucleic Acid Drug Dev 7(3): 187-95.

Toulme, J. J., R. L. Tinevez, et al. (1996). “Targeting RNA structures by antisense oligonucleotides.” Biochimie 78(7): 663-73.

### SUMMARY

The present disclosure provides, in various embodiments, a method of inhibiting the formation of a selected miRNA known to inhibit translation of one or more identified proteins, by exposing the cells to an antisense oligonucleotide complementary to a defined target region of the pri-miRNA precursor of the selected miRNA. In some embodiments, the antisense oligonucleotide compound is characterized by: (i) a substantially uncharged, nuclease-resistant backbone, (ii) capable of uptake into the nuclei of mammalian host cells, (iii) containing between 12-40 nucleotide bases, and (iv) having a targeting sequence of at least 12 contiguous bases complementary to a defined target region of the pri-miRNA precursor of the selected miRNA. The target region may be a 5′-end target region extending between the 5′-end nucleotide at which the pri-miRNA precursor is cleaved by Drosha and the nucleotide 30 bases upstream thereof, or a 3′-end target region extending between the 3′-end nucleotide at which the pri-miRNA precursor miRNA is cleaved by Drosha and the nucleotide 30 bases downstream thereof. In various embodiments, when the cells are exposed to the compound, there is formed a heteroduplex structure (i) composed of the pri-miRNA precursor and the oligonucleotide compound, and (ii) characterized by a Tm of dissociation of at least 45° C.

The oligonucleotide compound to which the host cells are exposed may be composed of morpholino subunits and phosphorus-containing intersubunit linkages joining a morpholino nitrogen of one subunit to a 5′ exocyclic carbon of an adjacent subunit. The morpholino subunits may be joined by intersubunit linkages having the structure:

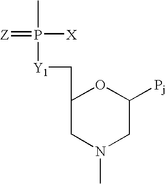

where Y₁=O, Z=O, Pj is a purine or pyrimidine base-pairing moiety effective to bind, by base-specific hydrogen bonding, to a base in a polynucleotide, and X is alkyl, alkoxy, thioalkoxy, amino or alkyl amino, including dialkylamino.

The antisense oligonucleotide may have a targeting sequence of at least 12 contiguous bases complementary to a sequence contained exclusively within the 5-end target sequence, and more specifically, a targeting sequence of at least 12 contiguous bases complementary to a target region contained exclusively within a region of the 5′-end target region between 8 and 25 nucleotides upstream of the nucleotide at which the pri-miRNA precursor miRNA is cleaved by Drosha.

In view of the involvement of miRNA in regulating numerous different mRNAs, antisense oligonucleotides directed against miRNAs can be used to treat a variety of disorders and disease conditions.

For use in treating glioblastomas or breast cancer in a human subject, the oligonucleotide compound may have a targeting sequence that may have at least 12 contiguous bases complementary to the target region identified by SEQ ID NO: 5 or 9. Exemplary oligonucleotide sequences targeting SEQ ID NO: 5 are SEQ ID NOS: 13-18, and for SEQ ID NO: 9, SEQ ID NOS: 19-23. The antisense oligonucleotide compound is administered to the human subject in a pharmaceutically acceptable dose.

For use in treating pediatric Burkitt's disease, Hodgkin lymphoma, primary mediastinal and diffuse large-B-cell lymphoma, or breast cancer in a human subject, the targeting sequence may have at least 12 contiguous bases complementary to the target region identified by SEQ ID NO: 6 or 10. Exemplary oligonucleotide sequences targeting SEQ ID NO: 6 are SEQ ID NOS: 34 and 35, and for SEQ ID NO: 10, SEQ ID NOS: 36 and 37. The antisense oligonucleotide compound is administered to the human subject in a pharmaceutically acceptable dose.

For use in treating hepatocellular carcinoma, or B-cell lymphoma in a human subject, the targeting sequence may have at least 12 contiguous bases complementary to the target region identified by SEQ ID NO: 7 or 11. Exemplary oligonucleotide sequences targeting SEQ ID NO: 7 are SEQ ID NOS: 38 and 39, and for SEQ ID NO: 11, SEQ ID NOS: 40 and 41. The antisense oligonucleotide compound is administered to the human subject in a pharmaceutically acceptable dose.

For use in treating leukemias of monocytic and myelocytic origin in a human, the targeting sequence may have 12 contiguous bases complementary to the target region of the pri-miRNA precursor of miR-223, and have sequences such as SEQ ID NOS: 45-47, targeting the region of the miR-223 pri-miRNA 5′ of the Drosha site, and SEQ ID NOS: 42-44 targeting the region of the miR-223 pri-miRNA 3′ of the Drosha site. The antisense oligonucleotide compound is administered to the human subject in a pharmaceutically acceptable dose.

For use in treating hyperlidipemia or a related cardiovascular disease in a human, the miRNA whose formation is inhibited may be miR-122a, the oligonucleotide compound may have a targeting sequence that is complementary to at least 12 contiguous bases of the sequence identified by SEQ ID NO: 8 or 12. Exemplary oligonucleotide sequences targeting SEQ ID NO: 8 are SEQ ID NOS: 24-28, and for SEQ ID NO: 12, SEQ ID NOS: 29-33. The antisense oligonucleotide compound is administered to the human subject in a pharmaceutically acceptable dose.

In some aspects, the disclosure includes a method of preparing a compound capable of inhibiting the formation of a selected miRNA known to inhibit translation of one or more identified proteins. In practicing the method, there is first identified one of (i) a 5′-end target sequence in the pri-miRNA precursor of the selected miRNA extending between the 5′-end nucleotide at which the pri-miRNA precursor is cleaved by Drosha and the nucleotide 30 bases upstream thereof, and (ii) a 3′-end target sequence in the pri-miRNA precursor extending between the 3′-end nucleotide at which the pri-miRNA precursor is cleaved by Drosha and the nucleotide 30 bases downstream thereof. An antisense oligonucleotide compound directed to the identified target sequence can then be prepared, as described below. In some embodiments, the antisense oligonucleotide is characterized by: (i) a substantially uncharged, nuclease-resistant backbone, (ii) capable of uptake into the nuclei of mammalian host cells, (iii) containing between 12-40 nucleotide bases, and (iv) having a targeting sequence of at least 12 contiguous bases complementary to the target sequence identified in step (a).

In some embodiments, the targeting sequence may contain at least 12 contiguous bases complementary to a sequence contained exclusively within the 5-end target sequence, and more specifically, may contain at least 12 contiguous bases complementary to the sequence contained exclusively within a region of the 5′-end target region between 8 and 25 nucleotides upstream of the nucleotide at which the pri-miRNA precursor miRNA is cleaved by Drosha.

In other aspects, the disclosure provides an antisense oligonucleotide compound for use in treating a cancer or hyperlipidemic condition in a human. In some embodiments, the compound is characterized by (i) a substantially uncharged, nuclease-resistant backbone, (ii) capable of uptake into the nuclei of mammalian host cells, (iii) containing between 12-40 nucleotide bases, and (iv) having a targeting sequence of at least 12 contiguous bases complementary to a target region selected from one of SEQ ID NOS: 5-7 or 9-11, for treating a human cancer, and SEQ ID NO: 8 or 12, for treating a hyperlipidemic condition. Exemplary pri-miRNA target and oligonucleotide targeting sequences are as given above.

In some embodiments, the oligonucleotide compound may be composed of morpholino subunits and phosphorus-containing intersubunit linkages joining a morpholino nitrogen of one subunit to a 5′ exocyclic carbon of an adjacent subunit, and the morpholino subunits may be joined by intersubunit linkages having the structure:

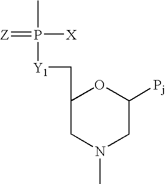

where Y₁=O, Z=O, Pj is a purine or pyrimidine base-pairing moiety effective to bind, by base-specific hydrogen bonding, to a base in a polynucleotide, and X is alkyl, alkoxy, thioalkoxy, amino or alkyl amino, including dialkylamino.

These and other objects and features of various embodiments will become more fully apparent when the following detailed description is read in conjunction with the accompanying drawings.

### BRIEF DESCRIPTION OF THE FIGURES

FIGS. 1A-1D show several preferred morpholino-type subunits having 5-atom (A), six-atom (B) and seven-atom (C-D) linking groups suitable for forming polymers.

FIGS. 2A-2G show examples of uncharged linkage types in oligonucleotide analogs. FIG. 2H shows a preferred positively charged linkage.

FIG. 3 shows the synthetic steps to produce subunits used to produce +PMO containing the (1-piperazino) phosphinylideneoxy cationic linkage as shown in FIG. 2H.

FIG. 4 shows the pre-miRNA stem-loop, mature miRNA sequence and the pri-miRNA target regions for miR-21 and miR-122a. The antisense oligomer target regions are underlined, the mature miRNA sequence is in italics and the Drosha cleavage sites are marked with arrows.

FIG. 5 shows the alignment of exemplary targeting oligomers of the invention with the pri-miRNA in relation to the Drosha cleavage sites (arrows) of pri-miR-21. Preferred targeting sequences are denoted with an asterisk.

FIG. 6 shows the decreased expression of mature miR-21 with respect to an endogenous control in cultured cells following treatment with the PMOs shown diagramatically in FIG. 5 and listed in Table 2 and the Sequence Listing (SEQ ID NOS: 3-13). The results are based on real-time quantitative PCR analysis of RNA extracted from HeLa cells treated with P008-conjugated PMOs.

### DETAILED DESCRIPTION

A. Definitions

The terms below, as used herein, have the following meanings, unless indicated otherwise:

The terms “antisense oligomer” or “antisense oligonucleotide” are used interchangeably and refer to a sequence of subunits, each having a base carried on a backbone subunit composed of ribose or other pentose sugar or morpholino group, and where the backbone groups are linked by intersubunit linkages that allow the bases in the compound to hybridize to a target sequence in a nucleic acid (typically an RNA) by Watson-Crick base pairing, to form a nucleic acid:oligomer heteroduplex within the target sequence. The oligomer may have exact sequence complementarity to the target sequence or near complementarity. Such antisense oligomers are designed to block or inhibit the biological activity of the RNA containing the target sequence, and may be said to be “targeted to” a sequence with which it hybridizes.

A “morpholino oligomer” refers to a polymeric molecule having a backbone which supports bases capable of hydrogen bonding to typical polynucleotides, wherein the polymer lacks a pentose sugar backbone moiety, and more specifically a ribose backbone linked by phosphodiester bonds which is typical of nucleotides and nucleosides, but instead contains a ring nitrogen with coupling through the ring nitrogen. A preferred “morpholino” oligomer is composed of morpholino subunit structures linked together by phosphoramidate or phosphorodiamidate linkages, joining the morpholino nitrogen of one subunit to the 5′ exocyclic carbon of an adjacent subunit, each subunit including a purine or pyrimidine base-pairing moiety effective to bind, by base-specific hydrogen bonding, to a base in a polynucleotide. Morpholino oligomers (including antisense oligomers) are detailed, for example, in co-owned U.S. Pat. Nos. 5,698,685, 5,217,866, 5,142,047, 5,034,506, 5,166,315, 5,185,444, 5,521,063, and 5,506,337, all of which are expressly incorporated by reference herein.

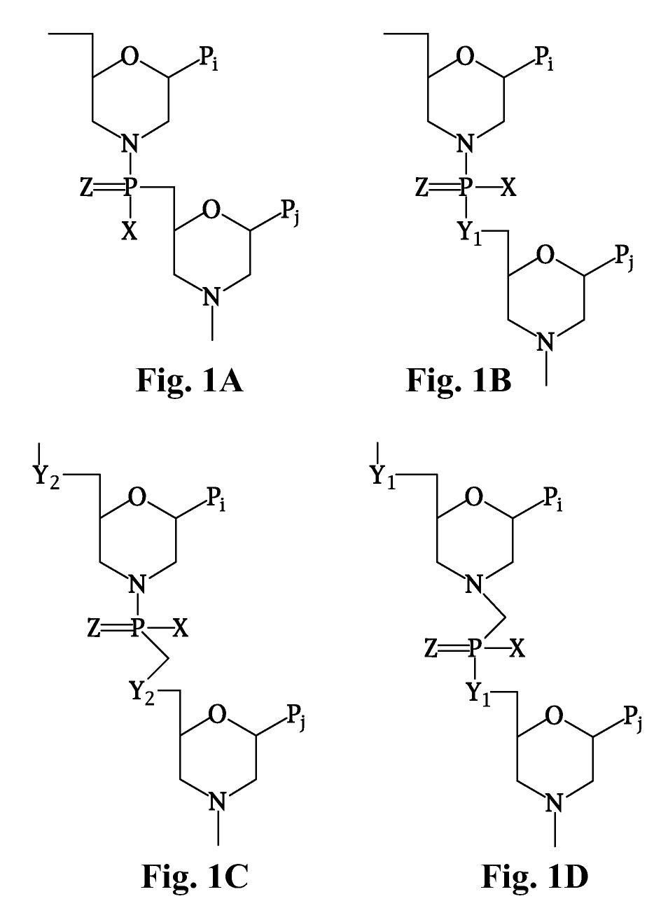

A phosphoramidate group comprises phosphorus having three attached oxygen atoms and one attached nitrogen atom, while a phosphorodiamidate group (see, e.g., FIGS. 1A-B) comprises phosphorus having two attached oxygen atoms and two attached nitrogen atoms. In the uncharged or the cationic intersubunit linkages of the oligomers described herein, one nitrogen is always pendant to the backbone chain. The second nitrogen, in a phosphorodiamidate linkage, is typically the ring nitrogen in a morpholino ring structure (see FIGS. 1A-B).

The terms “charged”, “uncharged”, “cationic” and “anionic” as used herein refer to the predominant state of a chemical moiety at near-neutral pH, e.g. about 6 to 8. Preferably, the term refers to the predominant state of the chemical moiety at physiological pH, that is, about 7.4.

An oligonucleotide or antisense oligomer “specifically hybridizes” to a target polynucleotide if the oligomer hybridizes to the target under physiological conditions, with a Tm greater than 37° C. The “Tm” of an oligomer is the temperature at which 50% hybridizes to a complementary polynucleotide. Tm is determined under standard conditions in physiological saline, as described, for example, in Miyada et al., Methods Enzymol. 154:94-107 (1987).

Polynucleotides are described as “complementary” to one another when hybridization occurs in an antiparallel configuration between two single-stranded polynucleotides. Complementarity (the degree that one polynucleotide is complementary with another) is quantifiable in terms of the proportion of bases in opposing strands that are expected to form hydrogen bonds with each other, according to generally accepted base-pairing rules.

A first sequence is an “antisense sequence” with respect to a second sequence if a polynucleotide whose sequence is the first sequence specifically binds to, or specifically hybridizes with, the second polynucleotide sequence under physiological conditions.

An agent is “actively taken up by cells” when the agent can enter the cell by a mechanism other than passive diffusion across the cell membrane. The agent may be transported, for example, by “active transport”, referring to transport of agents across a mammalian cell membrane by e.g. an ATP-dependent transport mechanism, or by “facilitated transport”, referring to transport of antisense agents across the cell membrane by a transport mechanism that requires binding of the agent to a transport protein or a cell penetrating peptide, which then facilitates passage of the bound agent across the membrane. Alternatively, the antisense compound may be formulated in a complexed form, such as an agent having an anionic backbone complexed with cationic lipids or liposomes, which can be taken into cells by an endocytotic mechanism. The analog also may be conjugated, e.g., at its 5′ or 3′ end, to an arginine-rich peptide, e.g., a peptide composed of arginine and other amino acids including the non-natural amino acids 6-aminohexanoic acid and beta-alanine. Exemplary arginine-rich delivery peptides are listed as SEQ ID NOS: 48-50. These exemplary arginine-rich delivery peptides facilitate transport into the target host cell as described (Moulton, Nelson et al. 2004; Nelson, Stein et al. 2005).

The terms “modulating expression”, “inhibition of expression”, “inhibition of biogenesis” and/or “antisense activity” refer to the ability of an antisense oligomer to either enhance or, more typically, reduce the expression of a given miRNA, by interfering with the expression or biogenesis of the miRNA. In the case of reduced miRNA expression, the antisense oligomer may directly block the maturation or biogenesis of an miRNA precursor or contribute to the accelerated breakdown of an miRNA precursor.

An “effective amount” or “therapeutically effective amount” refers to an amount of antisense oligomer administered to a mammalian subject, either as a single dose or as part of a series of doses, which is effective to produce a desired therapeutic effect, typically by inhibiting expression of a selected target nucleic acid sequence.

“Treatment” of an individual (e.g. a mammal, such as a human) or a cell is any type of intervention used in an attempt to alter the natural course of the individual or cell. Treatment includes, but is not limited to, administration of a pharmaceutical composition, and may be performed either prophylactically or subsequent to the initiation of a pathologic event or contact with an etiologic agent.

“MicroRNA” or “miRNA” refers to a single-stranded RNA of approximately 22-25 nucleotides in length, which is generated by the RNase-III-type enzyme Dicer from an endogenous transcript (pre-miRNA) that contains a local hairpin structure.

“MicroRNA biogenesis” or “miRNA biogenesis” refers to the RNA metabolic process that begins with the primary microRNA transcript (pri-miRNA) and, through cleavage by Drosha to create an intermediate precursor microRNA species (pre-miRNA) and subsequent processing by Dicer, ends with the mature miRNA.

“Drosha” refers to a nuclear RNase III enzyme that cuts pri-miRNA in a stem-loop portion of a double stranded RNA hairpin to generate precursor miRNA (pre-miRNA), which is approximately 60 nucleotides in length with a 3′ 2-nucleotide overhang. Drosha cleaves the stem loop in two sites, one proximal to the 5′ end of the stem loop; and the other, proximal to the 3′ end of the stem loop, and offset from the 5′ end site by 2-3 bases. The 5′-end nucleotide at which the pri-miRNA precursor is cleaved by Drosha is the nucleotide immediately adjacent the 5′-end nucleotide of the resulting pre-miRNA. The 3′-end nucleotide at which the pri-miRNA precursor miRNA is cleaved by Drosha is the nucleotide immediately adjacent the 3′ end nucleotide of the resulting pre-miRNA.

B. miRNA Biogenesis

Transcription of miRNA genes is mediated by RNA polymerase II (pol II) to produce primary transcripts (pri-miRNAs) that are sometimes several kilobases long. Pri-miRNA transcripts contain both a 5′ terminal cap structure and a 3′ terminal poly(A) tail. Several poly(A)-containing transcripts containing both miRNA sequences and regions of adjacent mRNAs have been characterized. The expression profiles of miRNA transcripts indicate that miRNA transcription is under elaborate control during development and in various tissues.

The maturation of miRNA appears to occur via two steps. First, miRNAs are transcribed as long primary transcripts (pri-miRNAs) that are first trimmed into hairpin intermediates called precursor miRNAs (pre-miRNAs) that are subsequently cleaved into mature miRNAs. The catalytic activities for the first and the second processing steps are compartmentalized into the nucleus and the cytoplasm, respectively. Furthermore, the nuclear export of pre-miRNA is necessary for cytoplasmic processing to occur. Transcription of miRNA genes results in pri-miRNA molecules that are typically several kilobases long and that contain a local hairpin structure. The stem-loop structure is cleaved by the nuclear RNase III enzyme Drosha to release the pre-miRNA molecules. Drosha is a large protein of approximately 160 kDa, and, in humans, forms an even larger complex of approximately 650 kDa known as the Microprocessor complex. The enzyme is a Class II RNAse III enzyme having double-stranded RNA binding domain (dsRBD). Because the enzyme binds to and cleaves the double-stranded stem portion of pri-miRNA, efforts have been made to block enzyme activity by disrupting the double-stranded structure across (spanning) the Drosha cutting site or within the double stranded region of the resulting pre-miRNA.

Surprisingly, it has been discovered that the strongest inhibition of pri-miRNA biogenesis, as evidenced by decreased expression of mature miRNA with respect to an endogenous control in cultured cells, is achieved by blocking a sequence region of the pri-miRNA that does not span the Drosha cut site, and may be spaced from the Drosha cut site by up to 8 nucleotide bases or more and does not overlap with sequence in the pre-miRNA formed by Drosha cutting.

Once the pre-miRNAs are exported to the cytoplasm, another RNase III enzyme called “Dicer” cleaves the pre-miRNA to produce the mature approximately 22 nucleotide miRNA. Mature miRNAs are incorporated into an effector complex known as the miRNA-containing RNA-induced silencing complex or miRISC. This is in contrast to the effector complex that contains siRNA known as RISC or siRISC. The approximately 22-nucleotide miRNA duplexes do not persist in the cell for long as one strand of this duplex rapidly disappears whereas the other strand remains as a mature miRNA.

The antisense oligomers described herein are capable of modulating miRNA biogenesis by inhibition of the pri-miRNA to pre-miRNA Drosha processing step. As indicated above, oligomers that target the regions 5′ of the 5′ Drosha cleavage site and 3′ of the 3′ Drosha cleavage site, i.e., sequences unique to pri-miRNA, and not overlapping the Drosha 5′ or 3′ cutting sites, and permitting sequences up to eight bases of more from the Drosha cutting site, were found to greatly diminish the presence of the mature miRNA in the cytoplasm. As discussed below, these antisense oligomers have both in vitro and in vivo applications. For example, if a particular miRNA is associated with a given disease state, e.g., induces apoptosis, cancer, detrimental metabolites, etc., an appropriate antisense oligomer that targets that miRNA's pri-miRNA precursor can be introduced into the cell in order to inhibit the biogenesis of the microRNA and reduce the damage. Furthermore, antisense oligomers described herein can be introduced into a cell or an animal to study the function of the miRNA. For example, the biogenesis of a miRNA in a cell or an animal can be inhibited with a suitable antisense oligomer. The function of the miRNA can be inferred by observing changes associated with inhibition of the miRNA in the cell or animal in order to inhibit the activity of the miRNA.

C. Antisense Oligomer Targets, Targeting Sequences and Inhibition of miRNA Biogenesis

1. pri-miRNA Targets and Method of Compound Preparation

The present disclosure is based on the discovery that enhanced inhibition of miRNA biogenesis can be achieved with an antisense oligonucleotide compound that (i) targets a region identified by 30 bases, preferably 25 bases, in a 5′ and 3′ direction from the Drosha cleavage sites that convert pri-miRNA to pre-miRNA and that flank the pre-miRNA sequence, and (ii) have physical and pharmacokinetic features which allow effective interaction between the antisense compound and the pri-miRNA target within host cells, e.g., are able to be taken up by cells and into the nuclear compartments within cells, and bind with a relatively high Tm to the target pri-miRNA.

In preparing the oligonucleotide compounds, there is first selected an miRNA known to inhibit translation of one or more identified proteins. For example, in preparing a compound for the treatment of a given cancer in human, an miRNA known to affect the level of translation of one or more given protein associated with that cancer is identified. Exemplary target miRNA's are human miR-21, miR-155, miR-17, and miR-223, which are related to human cancers, and miR-122a, which is related to hyperlipidemia and associated cardiovascular diseases in humans. Information on the sequence identity of several miRNAs, the proteins whose levels are affected by that miRNAs, and disease-related associations with those proteins, can be found in a variety of sources, e.g., Davis, S. et al., Nucleic Acids Res. 34(8):2294 (2006). For example, specific miRNAs that play a role in developmental regulation and cell differentiation in mammals, and in cardiogenesis have been identified (see Zhao, Y. et al., “Serum response factor regulates a muscle-specific microRNA that targets Hand2 during cardiogenesis,” Nature 436:214-220 (2005)) and lymphocyte development (see Chen, C. et al., “MicroRNAs modulate hematopoietic lineage differentiation,” Science 303:83-87 (2004)). A number of studies demonstrate a connection between miRNA and human cancer (see Calin, G. A. et al., “MicroRNA profiling reveals distinct signatures in B cell chronic lymphocytic leukemias,” Proc. Natl. Acad. Sci. USA 101:11755-11760 (2004); Calin, G. A. et al., “Human microRNA genes are frequently located at fragile sites and genomic regions involved in cancers”, Proc Natl. Acad. Sci. USA, 101:2999-3004 (2004); McManus, M. T., “MicroRNAs and cancer,” Semin. Cancer Biol., 13:253-258 (2003); Lu, J. et al., “MicroRNA expression profiles classify human cancers,” Nature 435:834-838 (2005); Hammond, S. M., “MicroRNAs as oncogenes,” Curr. Opin Genet. Dev., 16:4-9 (2005); and Volinia, S. et al., “A microRNA expression signature of human solid tumors defines cancer gene targets,” Proc. Natl. Acad. Sci. USA 103:2257-2261 (2006)).

Additional reports implicate roles for mammalian miRNAs in metabolic pathways (see Esau, C. et al., “MicroRNA-143 regulates adipocyte differentiation,” J. Biol. Chem. 279:52361-52365 (2004); Poy, M. N. et al., “A pancreatic inlet-specific microRNA regulates insulin secretion,” Nature, 432:226-230 (2004); Krutzfeldt, J. et al., “Silencing of microRNAs in vivo with ‘antagomirs,” Nature, 438:685-689 (2004); and Esau, C. et al., “miR-122 regulation of lipid metabolism revealed by in vivo antisense targeting,” Cell Metab., 3:87-98 (2006)). MiRNAs have also been shown to suppress (see Lecellier, C. H. et al., “A cellular microRNA mediates antiviral defense in human cells,” Science, 308:557-560 (2005)) and enhance (see Jopling, C. L. et al., “Modulation of hepatitis C virus RNA abundance by a liver-specific MicroRNA,” Science, 309:1577-1581 (2005)) levels of viral RNA in cells. Shahi et al., “Argonaute-a database for gene regulation by mammalian microRNAs,” Nucleic Acids Res., 34:D115-D118 (2006) provides a database of miRNAs, in particular from human, mouse and rat.

Once a target miRNA is selected, the miRNA and associated pri-miRNA sequences can be can be identified utilizing readily available miRNA databases such as miRBase (Lewis, Burge et al. 2005) available at website www.microrna.sanger.ac.uk/sequences/index.shtml and the human genome database at the NCBI (at website www.ncbi.nlm.nih.gov/genome/guide/human/). Sequence listings in miRBase often do not include sufficient pri-miRNA sequences to identify the target sequences, but do include either the known or putative pre-miRNA hairpin sequences, and GenBank database entries can be used to identify the known pri-miRNA sequences, Drosha cutting site, and target regions. That is, from the known human-genome sequence containing an identified miRNA sequence, the pri-miRNA sequences up to 30 bases 5′ to (upstream of) the 5′ end of the miRNA and up to 30 bases 3′ to (downstream of) the 3′-end of the miRNA can be identified, as targeting regions for the oligonucleotide compounds.

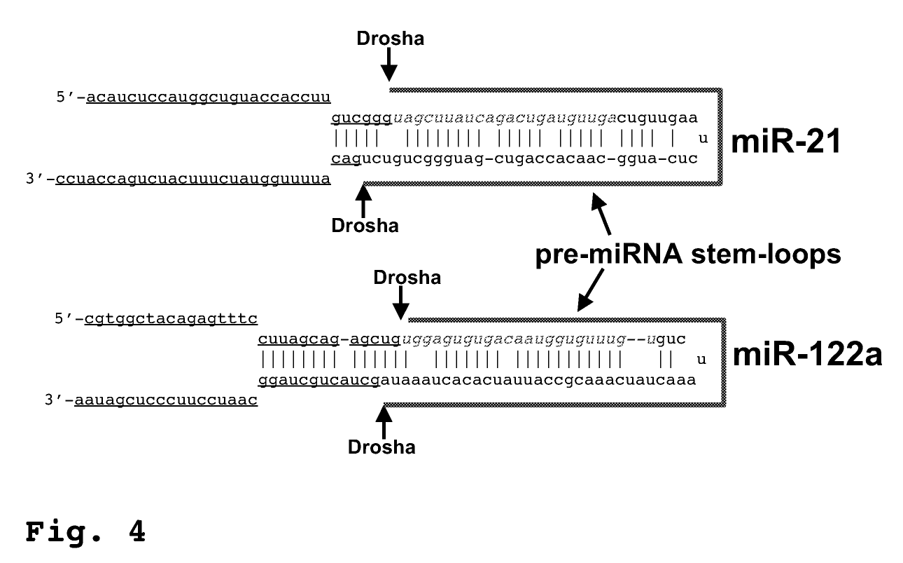

As examples, the sequences in Table 1 for the four identified miRNAs indicate the known or putative Drosha cleavage sites with a hyphen “-”. The 25 base target sequences on the 5′ and 3′ sides of the Drosha cleavage sites are underlined and also shown in the Sequence Listing as SEQ ID NOS: 5-8 for the target regions on the 5′ side of the 5′ Drosha cleavage site and SEQ ID NOS: 9-12 for the target regions on the 3′ side of the 3′ Drosha cleavage site. The miR-21 and miR-122a pri-miRNA sequences are shown in FIG. 4 in their predicted stem-loop form. FIG. 4 also has the target regions flanking the pre-miRNA stem-loop underlined and the Drosha cleavage sites marked with arrows. The predicted stem-loop sequences in miRBase may include the pre-miRNA and often some flanking sequence from the presumed pri-miRNA transcript. It will be appreciated that the sequences shown are expressed with DNA thymine bases (T) rather than the corresponding RNA uracil (U) bases. The actual pri-miRNA that is being targeted in contains uracil bases where thymine bases are indicated. Similarly, although the oligonucleotide targeting sequences, e.g., SEQ ID NOS: 13-47 below are indicated as containing thymine bases, the thymine bases may be substituted with uracil bases for complementarity to target adenine bases in the pri-miRNA, although thymine bases are generally employed in the oligonucleotides.

More generally, in preparing an antisense oligonucleotide compound for targeting a specific miRNA, one identifies either (i) the 5′-end target sequence in the pri-miRNA precursor of the selected miRNA extending between the 5′-end nucleotide at which the pri-miRNA precursor is cleaved by DROSHA and the nucleotide up to 30 bases, e.g., base 25, upstream thereof, or (ii) a 3′-end target sequence in the pri-miRNA precursor extending between the 3′-end nucleotide at which the pri-miRNA precursor is cleaved by Drosha and the nucleotide up to 30 bases, e.g., base 25, downstream thereof. The targeting sequence preferably excludes any overlap with miRNA sequences (across the Drosha cutting site), and may preferably be spaced up to 8 nucleotide bases or more from the Drosha cutting site. There is then selected a targeting sequence containing at least 12 contiguous bases complementary to this 5′ or 3′ pri-miRNA target sequence. An antisense compound having this targeting sequence can then be synthesized employing oligonucleotide structures and synthetic methods detailed herein. In some embodiments, the oligonucleotide synthesized is characterized by (i) a substantially uncharged, nuclease-resistant backbone, (ii) capable of uptake into the nuclei of mammalian host cells, and (iii) containing between 12-40 nucleotide bases.

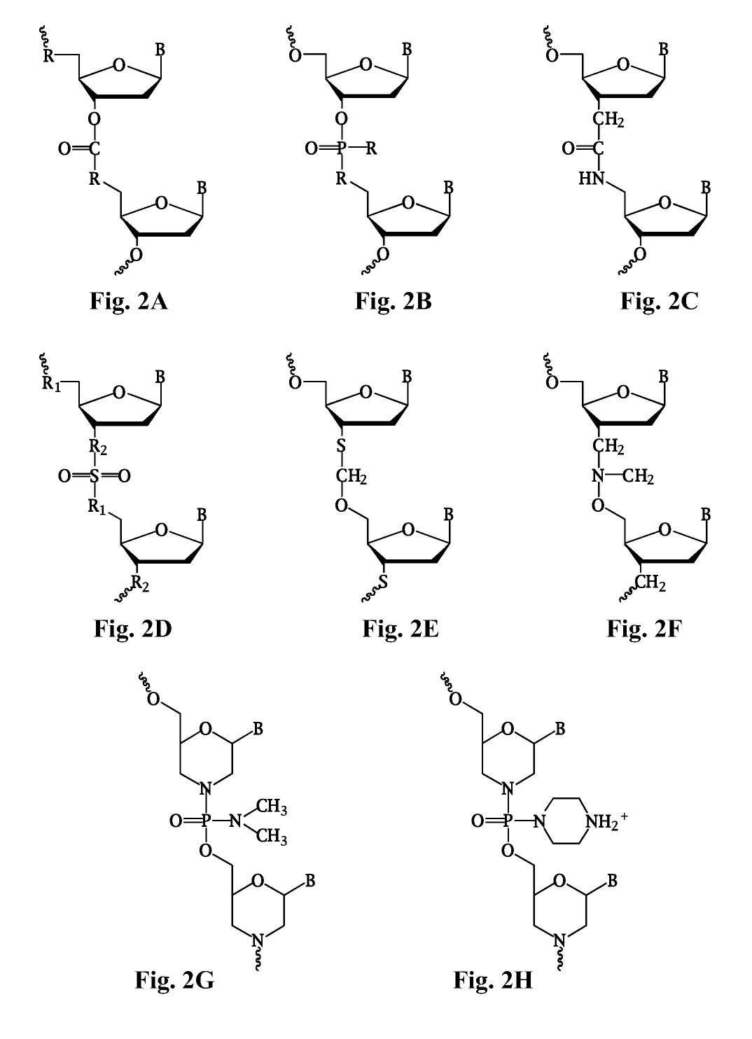

In some embodiments, the antisense oligonucleotide structure is composed of morpholino subunits and phosphorus-containing intersubunit linkages joining a morpholino nitrogen of one subunit to a 5′ exocyclic carbon of an adjacent subunit, where the morpholino subunits are joined by phosphorodiamidate linkages having the structure shown in FIG. 2G.

TABLE 1
Exemplary Human pri-miRNA Target Sequences

| | miRBase | | |
|---|---|---|---|
| | No. | | SEQ |
| Name | GenBank | | ID |
| (species) | No. | Sequence 5′-3′ | NO |
| miR-21 | MI0000077 | acatctccatggctgtaccacctt | 1 |
| (human) | AY699265 | gtcggg-tagcttatcagactgat | |
| | (2423-2543) | gttgactgttgaatctcatggcaa | |
| | | caccagtcgatgggctgtct-gac | |
| | | attttggtatctttcatctgacca | |
| | | tcc | |
| miR-155 | MI0000681 | ctgaaggcttgctgtaggctgtat | 2 |
| (human) | AF402776 | g-ctgttaatgctaatcgtgatag | |
| | (213-329) | gggtttttgcctccaactgactcc | |
| | | tacatattagcattaacagtg-ta | |
| | | tgatgcctgttactagcattcac | |
| miR-17 | MI0000071 | aagattgtgaccagtcagaataat | 3 |
| (human) | AB176708 | g-tcaaagtgcttacagtgcaggt | |
| | (1035-1146) | agtgatatgtgcatctactgcagt | |
| | | gaaggcacttgtagca-ttatggt | |
| | | gacagctgcctcgggaag | |
| miR-122a | MI0000442 | cgtggctacagagtttccttagca | 4 |
| (human) | AC105105 | gagctg-tggagtgtgacaatggt | |
| | (99166-99283) | gtttgtgtctaaactatcaaacgc | |
| | | cattatcacactaaata-gctact | |
| | | gctaggcaatccttccctcgataa | |

2. pri-miRNA Targeting Sequences

Generally, the degree of complementarity between the target and targeting sequence is sufficient to form a stable duplex. The region of complementarity of the antisense oligomers with the target RNA sequence may be as short as 8-11 bases, but is preferably 12-15 bases or more, e.g. 12-20 bases, or 12-25 bases. An antisense oligomer of about 14-15 bases is generally long enough to have a unique complementary sequence in the human transcriptome. In addition, a length of complementary bases sufficient to achieve the requisite binding Tₘ is discussed below. Oligomers as long as 40 bases may be suitable, where at least a sufficient number of bases, e.g., 12 bases, are complementary to the target sequence. In general, however, facilitated or active uptake in cells can be optimized at oligomer lengths less than about 30, preferably less than 25. For PMO oligomers, described further below, an optimum balance of binding stability and uptake generally occurs at lengths of 15-22 bases.

The oligomer may be 100% complementary to the pri-miRNA target sequence, or it may include mismatches, e.g., to accommodate variants, as long as a heteroduplex formed between the oligomer and viral nucleic acid target sequence is sufficiently stable to withstand the action of cellular nucleases and other modes of degradation which may occur in vivo. Oligomer backbones which are less susceptible to cleavage by nucleases are discussed below. Mismatches, if present, are less destabilizing toward the end regions of the hybrid duplex than in the middle. The number of mismatches allowed will depend on the length of the oligomer, the percentage of G:C base pairs in the duplex, and the position of the mismatch(es) in the duplex, according to well understood principles of duplex stability. Although such an antisense oligomer is not necessarily 100% complementary to the viral nucleic acid target sequence, it is effective to stably and specifically bind to the target sequence, such that a biological activity of the nucleic acid target, e.g., miRNA biogenesis, is modulated.

Generally, the stability of the duplex formed between the oligomer and the target sequence is a function of the binding Tₘ and the susceptibility of the duplex to cellular enzymatic cleavage. The Tₘ of an antisense compound with respect to complementary-sequence RNA may be measured by conventional methods, such as those described by Hames et al., Nucleic Acid Hybridization, IRL Press, 1985, pp. 107-108 or as described in Miyada C. G. and Wallace R. B., “Oligonucleotide hybridization techniques,” Methods Enzymol. Vol. 154:94-107 (1987). Each antisense oligomer should have a binding Tₘ, with respect to a complementary-sequence RNA, of greater than body temperature and preferably greater than 50° C. Tₘ's in the range 60-80° C. or greater are preferred. According to well known principles, the Tₘ of an oligomer compound, with respect to a complementary-based RNA hybrid, can be increased by increasing the ratio of C:G paired bases in the duplex, and/or by increasing the length (in base pairs) of the heteroduplex. At the same time, for purposes of optimizing cellular uptake, it may be advantageous to limit the size of the oligomer. For this reason, compounds that show high Tₘ (50° C. or greater) at a length of 25 bases or less may be used over those requiring greater than 25 bases for high Tₘ values.

The antisense activity of the oligomer may be enhanced by using a mixture of uncharged and cationic phosphorodiamidate linkages as shown in FIGS. 2G and 2H. The total number of cationic linkages in the oligomer can vary from 1 to 10, and be interspersed throughout the oligomer. In some embodiments, the number of charged linkages is at least 2 and no more than half the total backbone linkages, e.g., between 2-6 positively charged linkages, and preferably each charged linkages is separated along the backbone by at least one, preferably at least two uncharged linkages. The antisense activity of various oligomers can be measured in vitro by fusing the oligomer target region to the 5′ end a reporter gene (e.g. firefly luciferase) and then measuring the inhibition of translation of the fusion gene mRNA transcripts in cell free translation assays. The inhibitory properties of oligomers containing a mixture of uncharged and cationic linkages can be enhanced between, approximately, five to 100 fold in cell free translation assays.

Table 2 below shows exemplary targeting sequences, in a 5′-to-3′ orientation, that target the human pri-miRNAs of miR-21, miR-122a, miR-155, miR-17 and miR-223 (GenBank Acc. No. AY699265, NCBI36 Chromosome 18: 54269286-54269370 [+], AF402776, AB176708 and NCBI36 Chromosome X; 65155437-65155546 [+], respectively) according to the guidelines described above. The sequences listed provide a collection of targeting sequences from which individual targeting sequences may be selected, according to the general class rules discussed above. SEQ ID NOS:13-47 are antisense to the positive strand of the pri-miRNA.

TABLE 2

Exemplary Antisense Oligomer
Sequences Targeting Human pri-miRNAs

| | | SEQ |
|---|---|---|
| | | ID |
| Name | Sequence 5′-3′ | NO |
| miR-21-5′1 | CCC GAC AAG GTG GTA CAG CCA TGG | 13 |
| miR-21-5′2 | TGA TAA GCT ACC CGA CAA GG | 14 |
| miR-21-5′3 | CCC GAC AAG GTG GTA CAG | 15 |
| miR-21-5′4 | GGT GGT ACA GCC ATG GAG | 16 |
| miR-21-5′5 | TCA GTC TGA TAA GCT ACC C | 17 |
| miR-21-5′6 | GCT ACC CGA CAA GGT GGT ACA G | 18 |
| miR-21-3′1 | CAG ATG AAA GAT ACC AAA A | 19 |
| miR-21-3′2 | GAT GAA AGA TAC CAA AAT GTC | 20 |
| miR-21-3′3 | GAT ACC AAA ATG TCA GAC AGC C | 21 |
| miR-21-3′4 | TAG TCA GAC AGC CCA TCG ACT GG | 22 |
| miR-21-3′5c | CGA CTG GTG TTG CCA TGA GAT T | 23 |
| miR-122-5′1 | CAG CTC TGC TAA GGA AAC TCT GT | 24 |
| miR-122-5′2 | TCA CAC TCC ACA GCT CTG CT | 25 |
| miR-122-5′3 | CCA TTG TCA CAC TCC ACA G | 26 |
| miR-122-5′4 | GGA AAC TCT GTA GCC ACG AA | 27 |
| miR-122-5′5 | TAG CCA CGA AGG TGT TAA CT | 28 |
| miR-122-3′1 | AGG GAA GGA TTG CCT AGC A | 29 |
| miR-122-3′2 | TTG CCT AGC AGT AGC TAT TTA G | 30 |
| miR-122-3′3 | AGT AGC TAT TTA GTG TGA TAA TG | 31 |
| miR-122-3′4 | TGT GAT AAT GGC GTT TGA TAG T | 32 |
| miR-122-3′5 | GAC ATT TAT CGA GGG AAG GA | 33 |
| miR-155-5′1 | CAT ACA GCC TAC AGC AAG | 34 |
| miR-155-5′2 | CCT ACA GCA AGC CTT CAG | 35 |
| miR-155-3′1 | CTA GTA ACA GGC ATC ATA | 36 |
| miR-155-3′2 | GTG AAT GCT AGT AAC AGG | 37 |
| miR-17-5′1 | CAT TAT TCT GAC TGG TCA | 38 |
| miR-17-5′2 | CTG ACT GGT CAC AAT CTT | 39 |
| miR-17-3′1 | AGG CAG CTG TCA CCA TAA | 40 |
| miR-17-3′2 | AGG CAG CTG TCA CCA TAA | 41 |
| miR-223-3′1 | CTG GTA AGC ATG TGC CGC ACT T | 42 |
| miR-223-3′2 | CCG CAC TTG GGG TAT TTG AC | 43 |
| miR-223-3′3 | CCC TGG CCT AGA GCT GGT AAG | 44 |
| miR-223-5′1 | GTC AAA TAC ACG GAG CGT GGC | 45 |
| miR-223-5′2 | GAG CGT GGC ACT GCA GGA GGC | 46 |
| miR-223-5′3 | GTC CAA CTC AGC TTG TCA AAT A | 47 |

3. Inhibition of miRNA Biogenesis

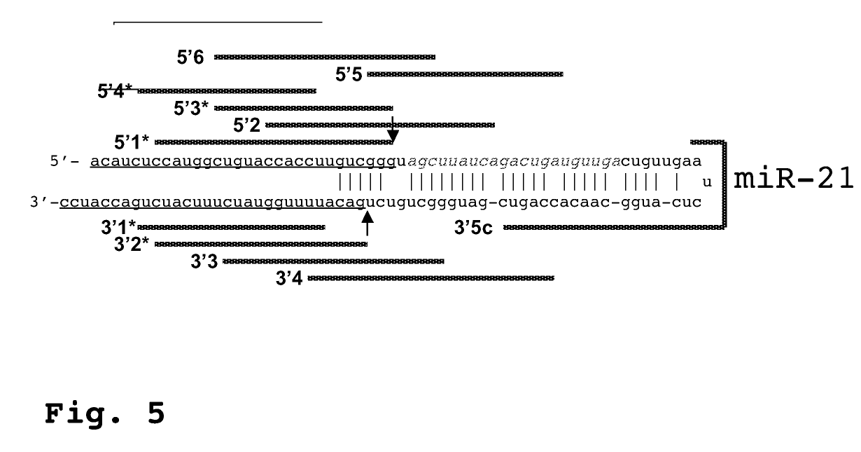

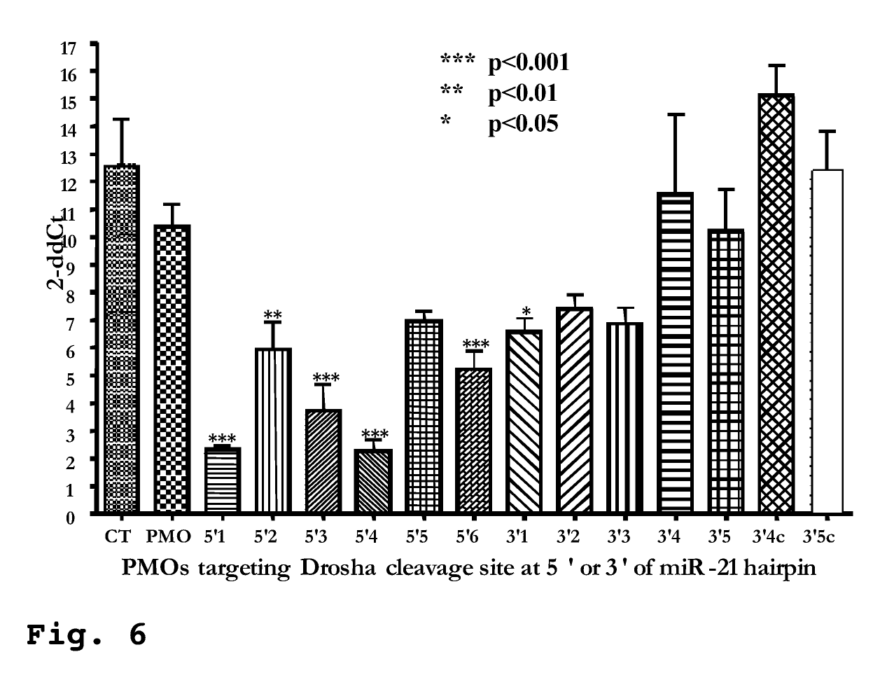

As described herein, antisense oligomers that target regions of pri-miRNA that flank the Drosha cleavage sites, relative to the pre-miRNA stem-loop, are found to have superior properties in the inhibition of miRNA biogenesis. FIG. 5 shows a targeting strategy used to investigate the ability of various PMO to inhibit the biogenesis of miR-21 in cell culture. PMOs were designed to target sequences that flank the Drosha cleavage sites (e.g., 5′1, 5′3, 5′4, 3′1 and 3′2; SEQ ID NOS: 3, 5, 6, 9 and 10, respectively), PMO that span the Drosha cleavage sites (e.g., 5′2, 5′5, 5′6, 3′3 and 3′4; SEQ ID NOS: 4, 7, 8, 11 and 12, respectively) and PMO that target only the pre-miRNA stem-loop (e.g., 3′5c; SEQ ID NO:13). As shown in FIG. 6, those PMO that do not span the Drosha cleavage site are equivalent to or significantly better at inhibition of miR-21 biogenesis than those that span the site. This is most apparent with PMOs that target regions flanking the 5′ cleavage site (e.g., compare 5′1 and 5′4 with 5′2) but also is seen at the 3′ cleavage site (e.g., compare 3′1 and 3′2 with 3′4).

Others have described inhibition of miRNA activity using anti-miRNA sequences (e.g., see Tuschl, et. al., WO2005079397A2; (Davis, Lollo et al. 2006; Esau, Davis et al. 2006) but these reports have not targeted antisense oligomers to the target sequences described herein. Instead, prior antisense targeting strategies have focused on either the mature miRNA molecule or the pre-miRNA stem-loop. The antisense oligomers described herein may exclude target sequences contained within the pre-miRNA molecules, and may even exclude sequence up to eight nucleotide bases away from the Drosha cutting site.

D. Antisense Oligonucleotide Analog Compounds

1 Properties

As detailed above, the antisense oligonucleotide analog compound (the term “antisense” indicates that the compound is targeted against the pri-miRNA coding sequence) has a base sequence target region that includes one or more of the following: 1) 30 bases in a 5′ direction from the 5′ Drosha cleavage sites, relative to the pre-miRNA sequence or; 2) 30 bases in a 3′ direction from the 3′ Drosha cleavage site, again relative to the pre-miRNA sequence. In addition, the oligomer is able to effectively target a pri-miRNA and prevent the processing by Drosha of the target pri-miRNA to its pre-miRNA form, when administered to a host cell, e.g. in a mammalian subject. This requirement may be met when the oligomer compound (a) has the ability to be actively taken up by mammalian cells and into the nuclear compartment, and (b) once taken up, form a duplex with the target RNA with a Tₘ greater than about 45° C.

As further described below, the ability of the oligonucleotide to be taken up by cells and into the nuclear compartment is observed when the oligomer backbone be substantially uncharged, and, preferably, that the oligomer structure is recognized as a substrate for active or facilitated transport across the cell membrane. The ability of the oligomer to form a stable duplex with the target RNA may also be influenced by the oligomer backbone, as well as factors noted above, e.g., the length and degree of complementarity of the antisense oligomer with respect to the pri-miRNA target, the ratio of G:C to A:T base matches, and the positions of any mismatched bases. The ability of the antisense oligomer to resist cellular nucleases promotes survival and ultimate delivery of the agent to the cell cytoplasm and nucleus.

Below are disclosed methods for testing any given, substantially uncharged backbone for its ability to display these properties.

2. Active or Facilitated Uptake by Cells

The antisense compound may be taken up by passive diffusion into host cells and into the cell's nuclear compartment, or by facilitated or active transport across the host cell membrane if administered in free (non-complexed) form, or by an endocytotic mechanism if administered in complexed form. In the latter case, the oligonucleotide compound may be a substrate for a membrane transporter system (i.e. a membrane protein or proteins) capable of facilitating transport or actively transporting the oligomer across the cell membrane. This feature may be determined by one of a number of tests for oligomer interaction or cell uptake, as follows.

A first test assesses binding at cell surface receptors, by examining the ability of an oligomer compound to displace or be displaced by a selected charged oligomer, e.g., a phosphorothioate oligomer, on a cell surface. The cells are incubated with a given quantity of test oligomer, which is typically fluorescently labeled, at a final oligomer concentration of between about 10-300 nM. Shortly thereafter, e.g., 10-30 minutes (before significant internalization of the test oligomer can occur), the displacing compound is added, in incrementally increasing concentrations. If the test compound is able to bind to a cell surface receptor, the displacing compound will be observed to displace the test compound. If the displacing compound is shown to produce 50% displacement at a concentration of 10× the test compound concentration or less, the test compound is considered to bind at the same recognition site for the cell transport system as the displacing compound.

A second test measures cell transport, by examining the ability of the test compound to transport a labeled reporter, e.g., a fluorescence reporter, into cells. The cells are incubated in the presence of labeled test compound, added at a final concentration between about 10-300 nM. After incubation for 30-120 minutes, the cells are examined, e.g., by microscopy, for intracellular label. The presence of significant intracellular label is evidence that the test compound is transported by facilitated or active transport.

In some embodiments, the antisense compound may also be administered in complexed form, where the complexing agent is typically a polymer, e.g., a cationic lipid, polypeptide, or non-biological cationic polymer, having an opposite charge to any net charge on the antisense compound. Methods of forming complexes, including bilayer complexes, between anionic oligonucleotides and cationic lipid or other polymer components, are well known. For example, the liposomal composition Lipofectin® (Felgner, Gadek et al. 1987), containing the cationic lipid DOTMA (N-[1-(2,3-dioleyloxy)propyl]-N,N,N-trimethylammonium chloride) and the neutral phospholipid DOPE (dioleyl phosphatidyl ethanolamine), is widely used. After administration, the complex is taken up by cells through an endocytotic mechanism, typically involving particle encapsulation in endosomal bodies.

In some embodiments, the antisense compound may also be administered in conjugated form with an arginine-rich peptide linked covalently to the 5′ or 3′ end of the antisense oligomer. The peptide is typically 8-16 amino acids and consists of a mixture of arginine, and other amino acids including phenylalanine and cysteine. The peptide may also contain non-natural amino acids such as beta-alanine and 6-aminohexanoic acid. Exemplary arginine-rich delivery peptides are listed as SEQ ID NOS: 49-50. The use of arginine-rich peptide-PMO conjugates to enhance cellular uptake of the antisense oligomer and methods of conjugating such peptides to a morpholino oligomer have been described. (See, e.g. (Moulton, Nelson et al. 2004; Nelson, Stein et al. 2005).

In some instances, liposomes may be employed to facilitate uptake of the antisense oligonucleotide into cells. (See, e.g., Williams, S. A., Leukemia 10(12):1980-1989 (1996); Lappalainen et al., Antiviral Res. 23:119 (1994); Uhlmann et al., “Antisense oligonucleotides: a new therapeutic principle,” Chemical Reviews 90(4):544-584 (1990); Gregoriadis, G., Chapter 14, Liposomes, Drug Carriers in Biology and Medicine, pp. 287-341, Academic Press, 1979). Hydrogels may also be used as vehicles for antisense oligomer administration, for example, as described in WO 93/01286. Alternatively, the oligonucleotides may be administered in microspheres or microparticles. (See, e.g., Wu, G. Y. and Wu, C. H., J. Biol. Chem. 262:4429-4432 (1987)). Alternatively, the use of gas-filled microbubbles complexed with the antisense oligomers can enhance delivery to target tissues, as described in U.S. Pat. No. 6,245,747.

Uptake into the nucleus of a cell can be monitored by conjugating a fluorescent tag (e.g., a fluorophore such as fluorescein) to the oligomer and then treating a target cell with the conjugate. In some embodiments, the conjugate can have a cell delivery peptide attached such as those described in the present disclosure. Treated cells can then be visualized using standard fluorescence microscopy or confocal microscopy to determine if the tagged oligomer has been transported to the nucleus.

Alternatively, in some embodiments, the requisite properties of oligomers with any given backbone can be confirmed by a simple in vivo test, in which a labeled compound is administered to an animal, and a body fluid sample, taken from the animal several hours after the oligomer is administered, assayed for the presence of heteroduplex with target RNA. This method is described in detail below.

3. Substantial Resistance to RNaseH

Two general mechanisms have been proposed to account for inhibition of expression by antisense oligonucleotides. (See e.g., (Agrawal, Mayrand et al. 1990; Bonham, Brown et al. 1995; Boudvillain, Guerin et al. 1997). In the first, a heteroduplex formed between the oligonucleotide and the viral RNA acts as a substrate for RNaseH, leading to cleavage of the viral RNA. Oligonucleotides belonging, or proposed to belong, to this class include phosphorothioates, phosphotriesters, and phosphodiesters (unmodified “natural” oligonucleotides). Such compounds expose the viral RNA in an oligomer:RNA duplex structure to hydrolysis by RNaseH, and therefore loss of function.

A second class of oligonucleotide analogs, termed “steric blockers” or, alternatively, “RNaseH inactive” or “RNaseH resistant”, have not been observed to act as a substrate for RNaseH, and are believed to act by sterically blocking target RNA nucleocytoplasmic transport, splicing or translation. This class includes methylphosphonates (Toulme, Tinevez et al. 1996), morpholino oligonucleotides, peptide nucleic acids (PNA's), certain 2′-O-allyl or 2′-O-alkyl modified oligonucleotides (Bonham, Brown et al. 1995), and N3′→P5′ phosphoramidates (Ding, Grayaznov et al. 1996; Gee, Robbins et al. 1998).

A test oligomer can be assayed for its RNaseH resistance by forming an RNA:oligomer duplex with the test compound, then incubating the duplex with RNaseH under standard assay conditions, as described by Stein, et. al. (Stein, Foster et al. 1997). After exposure to RNaseH, the presence or absence of intact duplex can be monitored by gel electrophoresis or mass spectrometry.

4. In vivo Uptake

In some embodiments, a simple, rapid test may be used for confirming that a given antisense oligomer type provides the characteristics noted above, namely, high Tₘ, ability to be actively taken up by the host cells and substantial resistance to RNaseH. This method is based on the discovery that a properly designed antisense compound will form a stable heteroduplex with the complementary portion of the target RNA when administered to a mammalian subject, and the heteroduplex subsequently appears in the urine (or other body fluid). Details of this method are given in co-owned U.S. patent application Ser. No. 09/736,920, entitled “Non-Invasive Method for Detecting Target RNA” (Non-Invasive Method), the disclosure of which is incorporated herein by reference.

Briefly, a test oligomer containing a backbone to be evaluated, and having a base sequence targeted against the target pri-miRNA RNA, is injected into a mammalian subject. Several hours (typically 8-72) after administration, the urine is assayed for the presence of the antisense-RNA heteroduplex. If heteroduplex is detected, the backbone is suitable for use in the antisense oligomers described herein.

The test oligomer may be labeled, e.g. by a fluorescent or a radioactive tag, to facilitate subsequent analyses, if it is appropriate for the mammalian subject. The assay can be in any suitable solid-phase or fluid format. Generally, a solid-phase assay involves first binding the heteroduplex analyte to a solid-phase support, e.g., particles or a polymer or test-strip substrate, and detecting the presence/amount of heteroduplex bound. In a fluid-phase assay, the analyte sample is typically pretreated to remove interfering sample components. If the oligomer is labeled, the presence of the heteroduplex is confirmed by detecting the label tags. For non-labeled compounds, the heteroduplex may be detected by immunoassay if in solid phase format or by mass spectroscopy or other known methods if in solution or suspension format.

When the antisense oligomer is complementary to a specific pri-miRNA target sequence, the method can be used to detect the presence of a given pri-miRNA during a treatment method.

5. Exemplary Oligomer Backbones

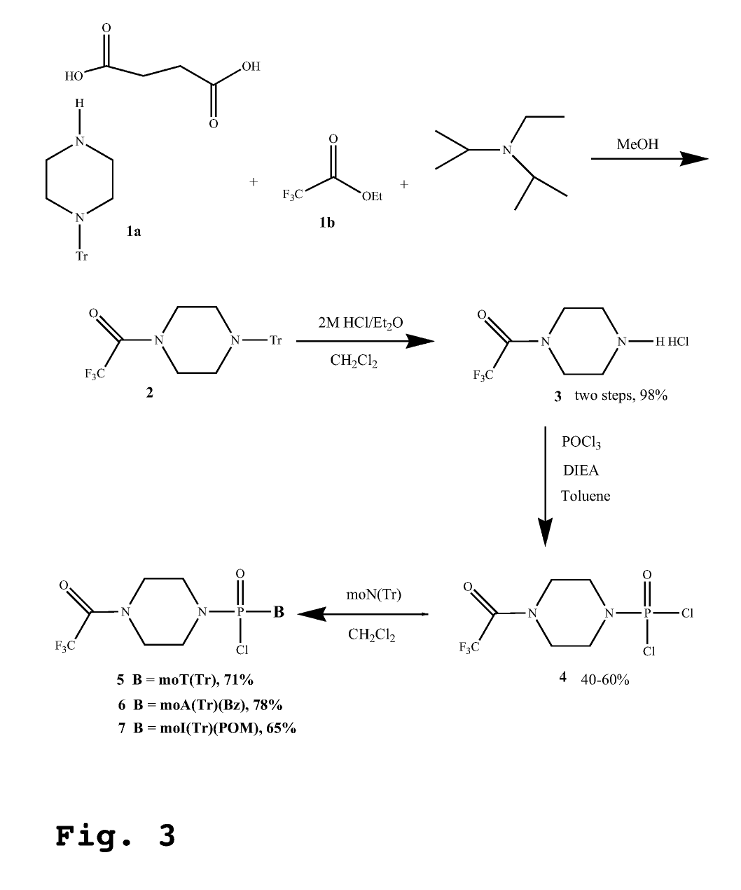

Examples of nonionic linkages that may be used in oligonucleotide analogs are shown in FIGS. 2A-2G. In these figures. FIG. 2B represents a purine or pyrimidine base-pairing moiety effective to bind, by base-specific hydrogen bonding, to a base in a polynucleotide, preferably selected from adenine, cytosine, guanine and uracil. Suitable backbone structures include carbonate (2A, R=O) and carbamate (2A, R=NH₂) linkages (Mertes and Coats 1969; Gait, Jones et al. 1974); alkyl phosphonate and phosphotriester linkages (2B, R=alkyl or —O-alkyl) (Lesnikowski, Jaworska et al. 1990); amide linkages (2C) (Blommers, Pieles et al. 1994); sulfone and sulfonamide linkages (2D, R₁, R₂=CH₂); and a thioformacetyl linkage (2E) (Cross, Rice et al. 1997). The latter is reported to have enhanced duplex and triplex stability with respect to phosphorothioate antisense compounds (Cross, Rice et al. 1997). Also reported are the 3′-methylene-N-methylhydroxyamino compounds of structure 2F. Also shown is a cationic linkage in FIG. 2H wherein the nitrogen pendant to the phosphate atom in the linkage of FIG. 2G is replaced with a 1-piperazino structure. The method for synthesizing the 1-piperazino group linkages is described below with respect to FIG. 3.

Peptide nucleic acids (PNAs) are analogs of DNA in which the backbone is structurally homomorphous with a deoxyribose backbone, consisting of N-(2-aminoethyl) glycine units to which pyrimidine or purine bases are attached. PNAs containing natural pyrimidine and purine bases hybridize to complementary oligonucleotides obeying Watson-Crick base-pairing rules, and mimic DNA in terms of base pair recognition (Egholm, Buchardt et al. 1993). The backbone of PNAs are formed by peptide bonds rather than phosphodiester bonds, making them well-suited for antisense applications. The backbone is uncharged, resulting in PNA/DNA or PNA/RNA duplexes which exhibit greater than normal thermal stability. PNAs are not recognized by nucleases or proteases.

In some embodiments, the oligomer structure employs morpholino-based subunits bearing base-pairing moieties, joined by uncharged linkages, as described above. Especially preferred is a substantially uncharged phosphorodiamidate-linked morpholino oligomer, such as illustrated in FIGS. 1A-1D, and in particular, in FIG. 2G. Morpholino oligonucleotides, including antisense oligomers, are detailed, for example, in co-owned U.S. Pat. Nos. 5,698,685, 5,217,866, 5,142,047, 5,034,506, 5,166,315, 5,185,444, 5,521,063, and 5,506,337, all of which are expressly incorporated by reference herein.

Important properties of the morpholino-based subunits include: the ability to be linked in a oligomeric form by stable, uncharged backbone linkages; the ability to support a nucleotide base (e.g., adenine, cytosine, guanine, thymidine, inosine or uracil) such that the polymer formed can hybridize with a complementary-base target nucleic acid, including target RNA, with high Tₘ, even with oligomers as short as 10-14 bases; the ability of the oligomer to be actively transported into mammalian cells; and the ability of the oligomer:RNA heteroduplex to resist RNAse degradation.

Exemplary backbone structures for antisense oligonucleotides include the β-morpholino subunit types shown in FIGS. 1A-1D, each linked by an uncharged, phosphorus-containing subunit linkage. FIG. 1A shows a phosphorus-containing linkage which forms the five atom repeating-unit backbone, where the morpholino rings are linked by a 1-atom phosphoamide linkage. FIG. 1B shows a linkage which produces a 6-atom repeating-unit backbone. In this structure, the atom Y linking the 5′ morpholino carbon to the phosphorus group may be sulfur, nitrogen, carbon or, preferably, oxygen. The X moiety pendant from the phosphorus may be fluorine, an alkyl or substituted alkyl, an alkoxy or substituted alkoxy, a thioalkoxy or substituted thioalkoxy, or unsubstituted, monosubstituted, or disubstituted nitrogen, including cyclic structures, such as morpholines or piperidines. Alkyl, alkoxy and thioalkoxy preferably include 1-6 carbon atoms. The Z moieties are sulfur or oxygen, and are preferably oxygen.

The linkages shown in FIGS. 1C and 1D are designed for 7-atom unit-length backbones. In Structure 1C, the X moiety is as in Structure 1B, and the moiety Y may be methylene, sulfur, or, preferably, oxygen. In Structure 1D, the X and Y moieties are as in Structure 1B. Particularly preferred morpholino oligonucleotides include those composed of morpholino subunit structures of the form shown in FIG. 1B, where X=NH₂ or N(CH₃)₂, Y=O, and Z=O. This preferred structure, as described, is also shown in FIG. 2G.

As noted above, the substantially uncharged oligomer may advantageously include a limited number of charged backbone linkages. One example of a cationic charged phophordiamidate linkage is shown in FIG. 2H. This linkage, in which the dimethylamino group shown in FIG. 2G is replaced a 1-piperazino group as shown in FIG. 2G, can be substituted for any linkage(s) in the oligomer. By including between two to eight such cationic linkages, and more generally, at least two and no more than about half the total number of linkages, interspersed along the backbone of the otherwise uncharged oligomer, antisense activity can be enhanced without a significant loss of specificity. The charged linkages are preferably separated in the backbone by at least 1 and preferably 2 or more uncharged linkages.

The antisense compounds can be prepared by stepwise solid-phase synthesis, employing methods detailed in the references cited above. In some cases, it may be desirable to add additional chemical moieties to the antisense compound, e.g. to enhance pharmacokinetics or to facilitate capture or detection of the compound. Such a moiety may be covalently attached, typically to a terminus of the oligomer, according to standard synthetic methods. For example, addition of a polyethyleneglycol moiety or other hydrophilic polymer, e.g., one having 10-100 monomeric subunits, may be useful in enhancing solubility. One or more charged groups, e.g., anionic charged groups such as an organic acid, may enhance cell uptake. A reporter moiety, such as fluorescein or a radiolabeled group, may be attached for purposes of detection. Alternatively, the reporter label attached to the oligomer may be a ligand, such as an antigen or biotin, capable of binding a labeled antibody or streptavidin. In selecting a moiety for attachment or modification of an antisense oligomer, it is generally of course desirable to select chemical compounds of groups that are biocompatible and likely to be tolerated by a subject without undesirable side effects.

A schematic of a synthetic pathway that can be used to make morpholino subunits containing a (1-piperazino) phosphinylideneoxy linkage is shown in FIG. 3; further experimental detail for a representative synthesis is provided in Materials and Methods, below. As shown in the figure, reaction of piperazine and trityl chloride gave trityl piperazine (1a), which was isolated as the succinate salt. Reaction with ethyl trifluoroacetate (1b) in the presence of a weak base (such as diisopropylethylamine or DIEA) provided 1-trifluoroacetyl-4-trityl piperazine (2), which was immediately reacted with HCl to provide the salt (3) in good yield. Introduction of the dichlorophosphoryl moiety was performed with phosphorus oxychloride in toluene.

The acid chloride (4) is reacted with morpholino subunits (moN), which may be prepared as described in U.S. Pat. No. 5,185,444 or in Summerton and Weller, 1997 (cited above), to provide the activated subunits (5,6,7). Suitable protecting groups are used for the nucleoside bases, where necessary; for example, benzoyl for adenine and cytosine, isobutyryl for guanine, and pivaloylmethyl for inosine. The subunits containing the (1-piperazino) phosphinylideneoxy linkage can be incorporated into the existing PMO synthesis protocol, as described, for example in Summerton and Weller (1997), without modification.

E. Treatment Method

In some embodiments, the antisense compounds detailed above are useful in inhibiting miRNA biogenesis in a mammalian subject. In this method, the oligonucleotide antisense compound can be administered to a mammalian subject, e.g., a human, in a suitable pharmaceutical carrier. The treatment method is intended to reduce a targeted miRNA level in the animal sufficiently to provide a therapeutic benefit, e.g., in the treatment of cancer, hyperlipidemia, or other condition affected by the levels of a given miRNA.

1. Administration of the Antisense Oligomer

Effective delivery of the antisense oligomer to the target nucleic acid can be effectuated by various techniques. In some embodiments, routes of antisense oligomer delivery include, but are not limited to, various systemic routes, including oral and parenteral routes, e.g., intravenous, subcutaneous, intraperitoneal, and intramuscular, as well as inhalation, transdermal and topical delivery. The appropriate route can be determined by one of skill in the art, as appropriate to the condition of the subject under treatment. For example, an appropriate route for delivery of an antisense oligomer in the treatment of a viral infection of the skin is topical delivery, while delivery of an antisense oligomer for the treatment of a viral respiratory infection is by inhalation. The oligomer may also be delivered directly to the site of viral infection, or to the bloodstream.

The antisense oligomer may be administered in any convenient vehicle which is physiologically acceptable. Such a composition may include any of a variety of standard pharmaceutically accepted carriers employed by those of ordinary skill in the art. Examples include, but are not limited to, saline, phosphate buffered saline (PBS), water, aqueous ethanol, emulsions, such as oil/water emulsions or triglyceride emulsions, tablets and capsules. The choice of suitable physiologically acceptable carrier will vary dependent upon the chosen mode of administration.

In some instances, liposomes may be employed to facilitate uptake of the antisense oligonucleotide into cells. (See, e.g., Williams, S. A., Leukemia 10(12):1980-1989 (1996); Lappalainen et al., Antiviral Res. 23:119 (1994); Uhlmann et al., “Antisense oligonucleotides: a new therapeutic principle,” Chemical Reviews, 90(4):544-584 (1990); Gregoriadis, G., Chapter 14, Liposomes, Drug Carriers in Biology and Medicine, pp. 287-341, Academic Press, 1979). Hydrogels may also be used as vehicles for antisense oligomer administration, for example, as described in WO 93/01286. Alternatively, the oligonucleotides may be administered in microspheres or microparticles. (See, e.g., Wu, G. Y. and Wu, C. H., J. Biol. Chem. 262:4429-4432 (1987)). Alternatively, the use of gas-filled microbubbles complexed with the antisense oligomers can enhance delivery to target tissues, as described in U.S. Pat. No. 6,245,747.

Sustained release compositions may also be used. These may include semipermeable polymeric matrices in the form of shaped articles such as films or microcapsules.

The antisense compound is generally administered in an amount and manner effective to result in a peak blood concentration of at least 200-400 nM antisense oligomer. Typically, one or more doses of antisense oligomer are administered, generally at regular intervals, for a period of about one to two weeks. Preferred doses for oral administration are from about 5-500 mg oligomer or oligomer cocktail per 70 kg individual. In some cases, doses of greater than 500 mg oligomer/subject may be necessary. For i.v. or i.p. administration, preferred doses are from about 1-250 mg oligomer or oligomer cocktail per 70 kg body weight. The antisense oligomer may be administered at regular intervals for a short time period, e.g., daily for two weeks or less. However, in some cases the oligomer is administered intermittently over a longer period of time. Administration may be followed by, or concurrent with, administration of an antibiotic or other therapeutic treatment. The treatment regimen may be adjusted (dose, frequency, route, etc.) as indicated, based on the results of immunoassays, other biochemical tests and physiological examination of the subject under treatment. Effective dosages and appropriate treatment regimen are well within the skill of those in the art given the knowledge in the art and the guidance provided in the present disclosure.

2. Treatment of Cancers

As indicated above, the present invention can be used both for designing oligonucleotide compounds capable of treating a selected cancer, and for treating the cancer by administering the compound in a therapeutic dose. In the treatment method, a human patient diagnosed with having a given cancer is administered a therapeutic amount of an oligonucleotide targeted against the pri-miRNA associated with that cancer. The patient may be receiving, or be placed on another chemotherapeutic agent, or treatment modality, such as x-ray therapy, concomitant with the present oligonucleotide treatment. The amount of oligonucleotide compound administered is as indicated above, and the ability of the compound to target the selected pri-miRNA may be monitored as above. Treatment may be continued according to a selected dosage regimen, e.g., once or twice weekly, until a desired improvement/remission is observed.

For use in treating glioblastomas or breast cancer in a human subject, the oligonucleotide compound may have a targeting sequence may have at least 12 contiguous bases complementary to the target region identified by SEQ ID NO: 5 or 9. Exemplary oligonucleotide sequences targeting SEQ ID NO: 5 are SEQ ID NOS: 13-18, and for SEQ ID NO: 9, SEQ ID NOS: 19-23.

For use in treating pediatric Burkitt's disease, Hodgkin lymphoma, primary mediastinal and diffuse large-B-cell lymphoma, or breast cancer in a human subject, the targeting sequence may have at least 12 contiguous bases complementary to the target region identified by SEQ ID NO: 6 or 10. Exemplary oligonucleotide sequences targeting SEQ ID NO: 6 are SEQ ID NOS: 34 and 35, and for SEQ ID NO: 10, SEQ ID NOS: 36 and 37.

For use in treating hepatocellular carcinoma, or B-cell lymphoma in a human subject, the targeting sequence may have at least 12 contiguous bases complementary to the target region identified by SEQ ID NO: 7 or 11. Exemplary oligonucleotide sequences targeting SEQ ID NO: 7 are SEQ ID NOS: 38 and 39, and for SEQ ID NO: 11, SEQ ID NOS: 40 and 41.

For use in treating leukemias of monocytic and myelocytic origin in a human, the targeting sequence may have 12 contiguous bases complementary to the target region of the pri-miRNA precursor of miR-223, and have sequences such as SEQ ID NOS: 45-47, targeting the region of the miR-223 pri-miRNA 5′ of the Drosha site, and SEQ ID NOS: 42-44 targeting the region of the miR-223 pri-miRNA 3′ of the Drosha site.

3. Treatment of Cardiovascular Disease

In some embodiments, the treatment methods can be used in treating hyperlipidemia, such as elevated levels of HDL or triglycerides, and cardiovascular disease, e.g., atherosclerosis associated with elevated levels of certain these lipids. The compound is preferably administered in an oral dose that can be taken on a daily basis, and may be monitored by standard lipid assays. The oligonucleotide compound may have a targeting sequence containing at least 12 contiguous bases complementary to the target region identified by SEQ ID NO: 8 or 12. Exemplary oligonucleotide sequences targeting SEQ ID NO: 8 are SEQ ID NOS: 24-28, and for SEQ ID NO: 12, SEQ ID NOS: 29-33. The antisense oligonucleotide compound is administered to the human subject in a pharmaceutically acceptable dose.

### EXAMPLES

A. Materials and Methods

All peptides were custom synthesized by Global Peptide Services (Ft. Collins, Colo.) or at AVI BioPharma (Corvallis, Oreg.) and purified to >90% purity. PMOs were synthesized at AVI BioPharma in accordance with known methods, as described, for example, in (Summerton and Weller 1997) and U.S. Pat. No. 5,185,444.

PMO oligomers were conjugated at the 5′end with one of two arginine-rich peptides (RAhxR)₄AhxβAla-5′-PMO or (RAhx)₈βAla-5′-PMO, SEQ ID NOS:48 and 49, respectively) to enhance cellular uptake and antisense activity as described (US Patent Publication 20040265879A1) and (Moulton, Nelson et al. 2004; Nelson, Stein et al. 2005). Beta-Alanine (βAla) and 6-aminohexanoic acid (Ahx) are non-natural amino acids.

B. Oligomer Synthesis

Preparation of N-trityl piperazine, succinate salt (1a): To a cooled solution of piperazine (10 eq) in toluene/methanol (5:1 toluene/methanol (v:v); 5 mL/g piperazine) was added slowly a solution of trityl chloride (1.0 eq) in toluene (5 mL/g trityl chloride). Upon reaction completion (1-2 hours), this solution was washed 4× with water. To the resulting organic solution was added an aqueous solution of succinic acid (1.1 eg; 13 mL water/g succinic acid). This mixture was stirred for 90 minutes, and the solid product was collected by filtration. The crude solid was purified by two reslurries in acetone. The yield was determined to be 70%.

Preparation of 1-trifluoroacetyl-4-trityl piperazine (2): To a slurry of 1a in methanol (10 mL/g 1a) was added diisopropylethylamine (2.1 eq) and ethyl trifluoroacetate (1.2 eq). After overnight stirring, the organic mixture was distilled to dryness. The resulting oil was dissolved in DCM (10 mL/g 1a) and washed 3× with 5% NaCl/H₂O. This solution was dried over Na₂SO₄, then concentrated to give a white foam. The yield was 100%.

Preparation of N-trifluoroacetyl piperazine, HCl salt (3): To a solution of 2 in DCM (10 mL/g 2) was added dropwise a solution of 2.0 M HCl/Et₂O (2.1 eq). The reaction mixture was stirred for 4 hours, and the product was collected by filtration. The filter cake was washed 3× with DCM. The solid was dried at 40° C. in a vacuum oven for 24 hours. Yield=95%. ¹⁹F NMR (CDCl₃)δ −68.2 (s); melting point=154-156° C.

Preparation of Activating Agent (4): To a cooled mixture of 3 (1.0 eq) and diisopropylethylamine (4.0 eq) in toluene (20 mL/g 3) was added slowly a solution of POCl₃ (1.1 eq) in toluene (20 mL/g 3). The reaction mixture was stirred in an ice bath for 4 hours. The reaction mixture was diluted with additional toluene (20 mL/g 3) and washed twice with 1 M KH₂PO₄ and once with 5% NaCl/H₂O. This solution was dried over Na₂SO₄ and distilled to an oil, which was then purified by silica gel chromatography (10% ethyl acetate/heptane as eluent). Yield was determined to be 50%.

Preparation of Activated Subunits (5, 6). To a cooled solution of 4 (1.2 eq) in DCM (10 mL/g 4) were added successively 2,6-lutidine (2.0 eq), N-methylimidazole (0.3 eq), and tritylated, base-protected (where necessary) morpholino subunit (1.0 eq). The solution was allowed to warm to room temperature. After 6 hours, the solution was washed with 1 M citric acid (pH 3). The organic layer was dried over Na₂SO₄, and the solvents were removed. The crude product was purified by silica gel chromatography (gradient of ethyl acetate/heptane). Yield was determined to be 60-70%.

C. Example 1 Inhibition of Human miR-21 Biogenesis in Tissue Culture

Expression of miR-21 in HeLa cells following treatment with peptide-conjugated PMOs was performed with a series of PMOs (Table 2; SEQ ID NOS: 13-23) that target various regions of the miR-21 pri-miRNA as shown in FIG. 5. The targeting strategy focused on PMOs that were entirely outside the pre-miRNA sequence and flanking either the 5′ or 3′ Drosha cleavage site, that spanned the Drosha cleavage site or that were entirely within the pre-miRNA stem-loop sequence. FIG. 4 shows the relationship of the pre-miRNA stem-loop to part of the pri-miRNA transcripts for miR-21 and miR-122a.

P008 peptide-conjugated PMOs (SEQ ID NO: 49 conjugated to the 5′end of the PMOs) were incubated with HeLa cells at a concentration of 2 micromolar for 72 hours. RNA was extracted and analyzed by quantitative real-time PCR for the mature miRNA product (miR-RT-PCR). The results are shown in FIG. 6 and plotted as the power ddCt for each PMO. This value represents the copy number of the miR-21 miRNA relative to an endogenous small nucleor control RNA (RNU-24). The ordinate value is therefore 2 to that power of that value (e.g., for the control, CT, it is 2^{12.5}). Therefore, the fold reduction of miR-21 after treatment with either the 5′1 and 5′4 PMOs compare to the control (CT) treatment is approximately 1450 fold. Compared to the three PMOs that span the 5′ Drosha cleavage site (5′2, 5'5 and 5′6; SEQ ID NOS: 14, 17 and 18), the three PMOs that target the flanking sequences (5′1, 5′3 and 5′4; SEQ ID NOS: 13, 15 and 16) are, on average, 13 times more effective in reducing the level of mature miR-21 in treated cells. A similar effect is observed for PMOs that target regions that either span or flank the 3′ Drosha cleavage site with flanking PMOs approximately 6 fold more effective. The 5′ flanking PMOs were overall more effective in inhibiting miR-21 biogenesis than those that target the 3′ flanking sequences for this particular miRNA.

All publications, patents, patent applications and other documents cited in this application are hereby incorporated by reference in their entireties for all purposes to the same extent as if each individual publication, patent, patent application or other document were individually indicated to be incorporated by reference for all purposes.

Although particular embodiments and applications have been described herein, it will be appreciated that a variety of changes and modifications can be made with departing from the spirit of the invention.

Sequence Listing

| | |
|---|---|
| | SEQ |
| | ID |
| Name | NO |

Target Sequences (5′ to 3′ )

| | | |
|---|---|---|
| miR-21 | ACATCTCCATGGCTGTACCACCTTGTCGGG- | 1 |
| (human) | TAGCTTATCAGACTGATGTTGACTGTTGAAT | |
| | CTCATGGCAACACCAGTCGATGGGCTGTCT- | |
| | GACATTTTGGTATCTTTCATCTGACCATCC | |
| miR-155 | CTGAAGGCTTGCTGTAGGCTGTATG- | 2 |
| (human) | CTGTTAATGCTAATCGTGATAGGGGT | |
| | TTTTGCCTCCAACTGACTCCTACATA | |
| | TTAGCATTAACAGTG-TATGATGCCT | |
| | GTTACTAGCATTCAC | |
| miR-17 | AGATTGTGACCAGTCAGAATAATG- | 3 |
| (human) | TCAAAGTGCTTACAGTGCAGGTAGTG | |
| | TATGTGCATCTACTGCAGTGAAGGC | |
| | CTTGTAGCA-TTATGGTGACAGCTG | |
| | CCTCGGGAAG | |
| miR-122a | CGTGGCTACAGAGTTTCCTTAGCAGAGCTG- | 4 |
| (human) | TGGAGTGTGACAATGGTGTTTGTGTCTAAAC | |
| | TATCAAACGCCATTATCACACTAAATA-GCT | |
| | CTGCTAGGCAATCCTTCCCTCGATAA | |
| miR-21-5′ | TCCATGGCTGTACCACCTTGTCGGG | 5 |
| miR-155-5′ | CTGAAGGCTTGCTGTAGGCTGTATG | 6 |
| miR-17-5′ | AGATTGTGACCAGTCAGAATAATG | 7 |
| miR-122a-5′ | CTACAGAGTTTCCTTAGCAGAGCTG | 8 |
| miR-21-3′ | GACATTTTGGTATCTTTCATCTGAC | 9 |
| miR-155-3′ | TATGATGCCTGTTACTAGCATTCAC | 10 |
| miR-17-3′ | TTATGGTGACAGCTGCCTCGGGAAG | 11 |
| miR-122a-3′ | GCTACTGCTAGGCAATCCTTCCCTC | 12 |
| | OLIGOMER TARGETING | |
| | SEQUENCES (5′ TO 3′) | |
| miR-21-5′1 | CCG GAC AAG GTG GTA CAG CCA TGG | 13 |
| miR-21-5′2 | TGA TAA GCT ACC CGA CAA GG | 14 |
| miR-21-5′3 | CCC GAC AAG GTG GTA CAG | 15 |
| miR-21-5′4 | GGT GGT ACA GCC ATG GAG | 16 |
| miR-21-5′5 | TCA GTC TGA TAA GCT ACC C | 17 |
| miR-21-5′6 | GCT ACC CGA CAA GGT GGT ACA G | 18 |
| miR-21-3′1 | CAG ATG AAA GAT ACC AAA A | 19 |
| miR-21-3′2 | GAT GAA AGA TAC CAA AAT GTC | 20 |
| miR-21-3′3 | GAT ACC AAA ATG TCA GAC AGC C | 21 |
| miR-21-3′4 | TAG TCA GAC AGC CCA TCG ACT GG | 22 |
| miR-21-3′5c | CGA CTG GTG TTG CCA TGA GAT T | 23 |
| miR-122-5′1 | CAG CTC TGC TAA GGA AAC TCT GT | 24 |
| miR-122-5′2 | TCA CAC TCC ACA GCT CTG CT | 25 |
| miR-122-5′3 | CCA TTG TCA CAC TCC ACA G | 26 |
| miR-122-5′4 | GGA AAC TCT GTA GCC ACG AA | 27 |
| miR-122-5′5 | TAG CCA CGA AGG TGT TAA CT | 28 |
| miR-122-3′1 | AGG GAA GGA TTG CCT AGC A | 29 |
| miR-122-3′2 | TTG CCT AGC AGT AGC TAT TTA G | 30 |
| miR-122-3′3 | AGT AGC TAT TTA GTG TGA TAA TG | 31 |
| miR-122-3′4 | TGT GAT AAT GGC GTT TGA TAG T | 32 |
| miR-122-3′5 | GAC ATT TAT CGA GGG AAG GA | 33 |
| miR-1 55-5′1 | CAT ACA GCC TAC AGC AAG | 34 |
| miR-1 55-5′2 | CCT ACA GCA AGC CTT CAG | 35 |
| miR-155-3′1 | CTA GTA ACA GGC ATC ATA | 36 |
| miR-1 55-3′2 | GTG AAT GCT AGT AAC AGG | 37 |
| miR-17-5′1 | CAT TAT TCT GAC TGG TCA | 38 |
| miR-17-5′2 | CTG ACT GGT CAC AAT CTT | 39 |
| miR-17-3′1 | AGG CAG CTG TCA CCA TAA | 40 |
| miR-17-3′2 | AGG CAG CTG TCA CCA TAA | 41 |
| miR-223-3′1 | CTG GTA AGC ATG TGC CGC ACT T | 42 |
| miR-223-3′2 | CCG CAC TTG GGG TAT TTG AC | 43 |
| miR-223-3′3 | CCC TGG CCT AGA GCT GGT AAG | 44 |
| miR-223-5′1 | GTC AAA TAC ACG GAG CGT GGC | 45 |
| miR-223-5′2 | GAG CGT GGC ACT GCA GGA GGC | 46 |
| miR-223-5′3 | GTC CAA CTC AGC TTG TCA AAT A | 47 |
| | Peptide Sequences (NHhd 2 to COOH) | |
| P007 | (RAhxR)₄AhxβAla | 48 |
| P008 | (RAhx)₈βAla | 49 |
| RB₇RXB | (RβAla)₇RAhxβAla | 50 |

What is claimed is:

1. A method of inhibiting the formation of a selected miRNA known to inhibit translation of one or more identified proteins, comprising:
(a) exposing the cells to an antisense oligonucleotide compound characterized by:
(i) a substantially uncharged, nuclease-resistant backbone,
(ii) capable of uptake into the nuclei of mammalian host cells,
(iii) containing between 12-40 nucleotide bases, and
(iv) having a targeting sequence of at least 12 contiguous bases complementary to a target region selected from one of: (i) a 5′-end target region extending between the 5′-end nucleotide at which the pri-miRNA precursor of the selected miRNA is cleaved by Drosha and the nucleotide 30 bases upstream thereof, and (ii) a 3′-end target region extending between the 3′-end nucleotide at which the pri-miRNA precursor miRNA is cleaved by Drosha and the nucleotide 30 bases downstream thereof,
(b) by the exposing, forming a heteroduplex structure (i) composed of the pri-miRNA precursor and the oligonucleotide compound, and (ii) characterized by a Tm of dissociation of at least 45° C.

2. The method of claim 1, wherein the oligonucleotide compound to which the host cells are exposed is composed of morpholino subunits and phosphorus-containing intersubunit linkages joining a morpholino nitrogen of one subunit to a 5′ exocyclic carbon of an adjacent subunit.

3. The method of claim 2, wherein the morpholino subunits in the oligonucleotide compound to which the host cells are exposed is administered to the subject are joined by intersubunit linkages having the structure: 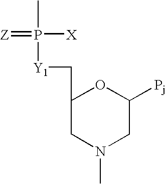
where Y₁=O, Z=O, Pj is a purine or pyrimidine base-pairing moiety effective to bind, by base-specific hydrogen bonding, to a base in a polynucleotide, and X is alkyl, alkoxy, thioalkoxy, amino or alkyl amino, including dialkylamino.

4. The method of claim 1, wherein the antisense oligonucleotide has a targeting sequence of at least 12 contiguous bases complementary to a sequence contained exclusively within the 5-end target sequence.

5. The method of claim 4, wherein the antisense oligonucleotide compound has a targeting sequence of at least 12 contiguous bases complementary to a target region contained exclusively within a region of the 5′-end target region between 8 and 25 nucleotides upstream of the nucleotide at which the pri-miRNA precursor miRNA is cleaved by Drosha.

6. The method of claim 1, for use in treating glioblastomas or breast cancer in a human subject, wherein the miRNA whose formation is inhibited is miR-21, and the antisense oligonucleotide compound is administered to the human subject in a pharmaceutically acceptable dose.

7. The method of claim 6, wherein the oligonucleotide compound has a targeting sequence that is complementary to at least 12 contiguous bases of the sequence identified by SEQ ID NO: 5.

8. The method of claim 1, for use in treating pediatric Burkitt's disease, Hodgkin lymphoma, primary mediastinal and diffuse large-B-cell lymphoma, or breast cancer in a human subject, wherein the miRNA whose formation is inhibited is miR-155, and the antisense oligonucleotide compound is administered to the human subject in a pharmaceutically acceptable dose.

9. The method of claim 8, wherein the oligonucleotide compound has a targeting sequence that is complementary to at least 12 contiguous bases of the sequence identified by SEQ ID NO: 6.

10. The method of claim 1, for use in treating hepatocellular carcinoma, or B-cell lymphoma, in a human subject, wherein the miRNA whose formation is inhibited is miR-17, and the antisense oligonucleotide compound is administered to the human subject in a pharmaceutically acceptable dose.

11. The method of claim 10, wherein the oligonucleotide compound has a targeting sequence that is complementary to at least 12 contiguous bases of the sequence identified by SEQ ID NO: 7.

12. The method of claim 1, for use in treating hyperlidipemia or a related cardiovascular disease in a human, wherein the miRNA whose formation is inhibited is miR-122a, and the antisense oligonucleotide compound is administered to the human subject in a pharmaceutically acceptable dose.

13. The method of claim 10, wherein the oligonucleotide compound has a targeting sequence that is complementary to at least 12 contiguous bases of the sequence identified by SEQ ID NO: 8.

14. A method of preparing a compound capable of inhibiting the formation of a selected miRNA known to inhibit translation of one or more identified proteins, comprising:
(a) identifying one of (i) a 5′-end target sequence in the pri-miRNA precursor of the selected miRNA extending between the 5′-end nucleotide at which the pri-miRNA precursor is cleaved by DROSHA and the nucleotide 30 bases upstream thereof, and (ii) a 3′-end target sequence in the pri-miRNA precursor extending between the 3′-end nucleotide at which the pri-miRNA precursor is cleaved by Drosha and the nucleotide 30 bases downstream thereof, and
(b) preparing an antisense oligonucleotide compound characterized by:
(i) a substantially uncharged, nuclease-resistant backbone,
(ii) capable of uptake into the nuclei of mammalian host cells,
(iii) containing between 12-40 nucleotide bases, and
(iv) having a targeting sequence of at least 12 contiguous bases complementary to the target sequence identified in step (a).

15. The method of claim 14, wherein the targeting sequence in step (b) contains at least 12 contiguous bases complementary to a sequence contained exclusively within the 5-end target sequence.

16. The method of claim 15, wherein the targeting sequence in step (b) contains at least 12 contiguous bases complementary to the sequence contained exclusively within a region of the 5′-end target region between 8 and 25 nucleotides upstream of the nucleotide at which the pri-miRNA precursor miRNA is cleaved by Drosha.

17. An antisense oligonucleotide compound for use in treating a cancer or hyperlipidemic condition in a human, the compound being characterized by:
(i) a substantially uncharged, nuclease-resistant backbone,
(ii) capable of uptake into the nuclei of mammalian host cells,
(iii) containing between 12-40 nucleotide bases, and
(iv) having a targeting sequence of at least 12 contiguous bases complementary to a target region selected from one of SEQ ID NOS: 5-7 and 9-11, for treating a human cancer, and SEQ ID NOS: 8 and 12, for treating a hyperlipidemic condition.

18. The oligonucleotide compound of claim 17, for use in treating glioblastomas or breast cancer in a human subject, wherein the targeting sequence of step (b) has at least 12 contiguous bases complementary to the target regions identified by SEQ ID NOS: 5 and 9.

19. The oligonucleotide compound of claim 17, for use in treating pediatric Burkitt's disease, Hodgkin lymphoma, primary mediastinal and diffuse large-B-cell lymphoma, or breast cancer in a human subject, wherein the targeting sequence of step (b) has at least 12 contiguous bases complementary to the target regions identified by SEQ ID NOS: 6 and 10.

20. The oligonucleotide compound of claim 17, for use in treating hepatocellular carcinoma or B-cell lymphoma in a human subject, wherein the targeting sequence of step (b) has at least 12 contiguous bases complementary to the target regions identified by SEQ ID NOS: 7 and 11.

21. The oligonucleotide compound of claim 17, for use in treating hyperlipidemia or a related cardiovascular disease in a human subject, wherein the targeting sequence of step (b) has at least 12 contiguous bases complementary to the target regions identified by SEQ ID NOS: 8 and 12.

22. The oligonucleotide compound claim 17, which is composed of morpholino subunits and phosphorus-containing intersubunit linkages joining a morpholino nitrogen of one subunit to a 5′ exocyclic carbon of an adjacent subunit.

23. The oligonucleotide compound of claim 22, wherein the morpholino subunits are joined by intersubunit linkages having the structure: 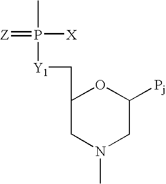
where Y₁=O, Z=O, Pj is a purine or pyrimidine base-pairing moiety effective to bind, by base-specific hydrogen bonding, to a base in a polynucleotide, and X is alkyl, alkoxy, thioalkoxy, amino or alkyl amino, including dialkylamino.
